(19) United States
(12) Patent Application Publication
(10) Pub. No.: US 2025/0083146 A1
(43) Pub. Date: Mar. 13, 2025

(54) NUCLEIC ACID TESTING IN A PORTABLE DEVICE

(71) Applicant: THE PENN STATE RESEARCH FOUNDATION, University Park, PA (US)

(72) Inventors: Weihua Guan, State College, PA (US); Tianyi Liu, State College, PA (US); Anthony J. Politza, University Park, PA (US)

(21) Appl. No.: 18/847,871

(22) PCT Filed: Mar. 21, 2023
(86) PCT No.: PCT/US2023/015783

§ 371 Date: Sep. 17, 2024

Related U.S. Application Data
(60) Provisional application No. 63/322,395, filed on Mar. 22, 2022.
(60) Provisional application No. 63/344,799, filed on May 23, 2022.

(51) Int. Cl. B01L 3/00

(52) U.S. Cl. CPC B01L 3/502761; B01L 3/502715; B01L 2200/0684; B01L 2200/14; B01L 2200/16; B01L 2300/1827; B01L 2400/043

(57) ABSTRACT

A nucleic acid testing device includes a microfluidic cartridge, an analyzer and a processing unit for controlling the analyzer. The microfluidic cartridge includes a binding chamber, a washing chamber and a reaction chamber. The analyzer includes a mechanical module including an electromagnetic coil array and a permanent magnet. The electromagnetic coil array is programmed to generate a programmable pulsed electromagnetic field to actuate the permanent magnet that further controls a paramagnetic bead (PMB) assay in the cartridge to move among the adjacent chambers.

### CROSS-REFERENCES TO RELATED APPLICATIONS

This application claims priority from U.S. Provisional Patent Application Ser. No. 63/322,395, filed on Mar. 22, 2022, and U.S. Provisional Patent Application Ser. No. 63/344,799, filed May 23, 2022, the entire content of both are incorporated herein by reference in their entirety.

### GOVERNMENT SUPPORT

This invention was made with government support under Grant No AI147419 awarded by the National Institutes of Health and under Grant Nos. ECCS1912410, ECCS2045169 and CBET1902503 awarded by the National Science Foundation. The Government has certain rights in the invention.

### FIELD OF THE INVENTION

The present invention relates to automated nucleic acid extraction and testing facilitated by a programmable electromagnetic (EM) pulse method to actuate a permanent magnet that controls a paramagnetic bead (PMB) assay in the cartridge.

### BACKGROUND OF THE INVENTION

Nucleic acid testing (NAT) remains the clinical standard for identification and quantification of infectious diseases. However, the laboratory procedures for these methods require long waiting periods, trained staff, and expensive hardware to analyze the testing results. Point-of-care (POC) devices developed over the last 10 or more years have started to introduce NAT in regions of need by simplifying the steps of NAT into compact and portable form-factors. However, the recent technological advancements motivating these devices have come in amplification and detection systems. While these developments are extremely important for the future of NAT devices, a bottleneck remains with sample preparation. Common sample preparation methods require extensive manual processes, laboratory devices, toxic chemicals, and trained professionals. These issues severely limit the scope of POC devices when attempting to provide quality NATs.

Numerous sample preparation systems for laboratories and POC devices depend on silica columns, membranes, or organic solvent methods. In laboratory settings these methods work well as they can be coupled with commercial devices and trained professionals, but they lack simple integration for POC applications. An alternative, coated paramagnetic beads (PMBs), offer a solution to these issues by eliminating the need for noxious chemicals and laboratory centrifuges. Sample preparation systems taking advantage of these PMBs have shown success in extracting and detecting nucleic acids for infectious diseases such as SARS-COV-2, HPV, HIV, RSV and genomic DNA. Even more applicable are lab-on-chip extraction systems. These systems paired with permanent magnets, rotational motors, and fluid pumps take advantage of the many ways that magnetic bead bound nucleic acids can be manipulated.

Lab-on-chip systems are appealing for POC applications because of their small form factor, reduced solution volumes, and disposability. Devices for POC applications adapt to lab-on-chip systems in order to handle the chip. There are devices integrating moving permanent magnets, electromagnet (EM) controls, EM actuated magnets, centrifuge style rotations, automated pipetting, and fluid flow methods. These devices integrate the control of the device into automated programs and lessen the need for user interference during the extraction process. Therefore, POC devices inherently increase the repeatability of testing, compared to lab-on-chip methods alone. On the other hand, these devices have areas of improvement for POC applications. Multiple devices cannot leave the laboratory because the setup is complex, or the device is large. Other devices are not truly automated, they still require intermittent manual steps. Even more downsides to these devices are linked to their lab-on-chip counterparts. Many microfluidic cartridges still require multiple manual steps, trained professionals for handling solutions, or fall into the “chip-in-lab” designation that cannot be used outside of the lab. This creates a need for a device that is fully automated, user friendly, ultra-portable, notably accessible, and sample adaptable.

The rapid, low-cost, easy-to-use NAT-on-USB would also be useful for the high-risk populations seeking private, highly sensitive self-testing at home.

According to the World Health Organization (WHO), HIV continues to be a significant global public health issue, having claimed 36.3 million lives so far (WHO 2021). Early and accurate HIV diagnosis is a critical step to initiate timely antiretroviral therapy (ART), which could suppress HIV, stop the progression of HIV disease, and reduce the viral load (VL) to undetectable levels (Zolopa 2010). The Joint United Nations Program on HIV/AIDS (UNAIDS) has thus put forth the ambitious goal to end AIDS as a global public health threat by 2030. This goal will highly depend on the increases in HIV testing, treatment, and viral suppression to prevent the onward transmission of HIV (Iwuji and Newell 2017). To this end, HIV self-testing is proposed as a new approach where an individual who wants to know HIV status collects a specimen, performs a test, and interprets the result privately (Parekh et al. 2018; Spielberg et al. 2004). In recent years, uptake of HIV self-testing has gained increasing acceptance both in the US and internationally (Frith 2007; Frye and Koblin 2017; Johnson and Corbett 2016; Ng and Tan 2013; Spielberg et al. 2004).

Existing HIV self-testing methods rely exclusively on widely adopted RDTs to detect the presence of HIV-1/2 antibodies (Fund 2022). While HIV RDT is very well suited for the primary screening process due to its low cost and fast turnaround time (de la Fuente et al. 2012; Mugo et al. 2017; Ng ct al. 2012; Sarkar et al. 2016), it could miss a significant portion of asymptomatic HIV carriers during the 2-4 weeks of the window period (Parekh et al. 2018; Stone et al. 2018). A possible alternative is to use nucleic acid testing (NAT), one of the most sensitive methods available for identifying the presence of HIV RNA and/or DNA (Parekh et al. 2018). NAT devices for HIV testing are readily available in centralized labs. However, a NAT device suitable for HIV self-testing is still lacking. In a recent report (Mazzola and Pérez-Casas 2015), WHO surveyed a list of HIV detection platforms such as Aptima HIV-1 Quant Assay (Hologic), GeneXpert HIV-1 Viral Load Test (Cepheid), Alere q system (Alere), cobas LiatTM System (Roche), and EOSCAPE-HIVTM HIV Rapid RNA Assay system (Wave 80 Biosciences). Most of these systems rely on relatively complex and expensive analyzers and replace conventional real-time PCR machines with portable thermal cyclers (Mauk et al. 2017). They often require plasma as a testing specimen which is prepared from venipuncture whole blood in laboratory conditions. Thus these NAT devices are not well suited for self-testing, in which a self-obtainable sample type such as finger-prick whole blood (Bertagnolio et al. 2010; Fidler et al. 2017; Guichet et al. 2018) or oral fluid would be preferred. To make the technologically intense HIV NATs more readily available in the resource-limited setting such as self-testing, there are increasing efforts in developing alternative isothermal amplification techniques that do not require thermal cycling and expensive instrumentation (Choi et al. 2018; Choi et al. 2016; Curtis et al. 2016; Curtis et al. 2012: Damhorst et al. 2015; Liu et al. 2011; Mauk et al. 2017; Myers et al. 2013; Phillips et al. 2018; Safavich et al. 2016; Singleton et al. 2014). These assays include loop-mediated isothermal amplification (LAMP), nucleic acid sequence-based amplification (NASBA), recombinase polymerase amplification (RPA), as well as helicase-dependent amplification (HIDA). Among isothermal methods. LAMP has been observed to be more resistant than PCR to inhibitors in complex samples such as blood (Wang et al. 2014). HIV LAMP assay (Curtis et al. 2014; Curtis et al. 2008, 2009; Ocwicja et al. 2015; Odari et al. 2015; Rudolph et al. 2015) has enabled the recent development of point-of-care HIV NAT devices, such as Smart Cup (Liao et al. 2016) and microRAAD (Phillips et al. 2019). Despite significant progress, no HIV NAT technologies can be used by a layperson to perform self-testing due to the complexity in sample handling (Dineva et al. 2007).

An ideal HIV self-test should combine the benefits of RDTs (minimal training, minimal sample handling, and rapid) and NAT (highly sensitive, specific, and quantitative capability). To this end, it would require a fully integrated sample preparation by automating the assay process with a cost-effective microfluidic chip and analyzer.

### SUMMARY OF THE INVENTION

The embodiments of the present invention provide an automated nuclei acid testing (NAT) device. The NAT device simplifies the process of sample preparation by automating nucleic acid extraction.

The NAT device may include a microfluidic reagent cartridge and an analyzer both housed in a housing. The cartridge includes a binding chamber, a washing chamber and a reaction or elution chamber. Optionally, there may be a valve chamber between the functional chambers. The valve chamber may be oil valves. The oils could mineral oil or fluoridated oil. There may be one or more valve chambers between the binding chamber and the washing chamber. There may be one or more valve chambers between the washing chamber and the reaction chamber. The cartridge may be preloaded with all reagents needed for operating a paramagnetic bead (PMB) assay. For actuating the nucleic acid-bearing magnetic beads in the cartridge, the analyzer may use a magnetic robot. The magnetic robot includes a double-sided planar electromagnetic coil array on a printed circuit board (PCB) paired with a permanent magnet. This PCB coil can be programmed to generate a localized pulsed electromagnetic field for actuating the permanent magnet that further controls the magnetic beads in the cartridge. The electromagnetic coils and permanent magnet may be located above the cartridge or below the cartridge. The electromagnetic coil array can be a one-dimensional (1D) array or a 2-dimensional (2D) array. For a 2D array, the electromagnet design contains groups and blocks of coils that are actuated in series. In one embodiment, each block holds three groups and each group have a group designation. The coils with a same group designation are interconnected across blocks.

In the binding chamber, the nucleic acids bind with the PMBs. The binding buffer decreases the pH of the solution to a more ideal condition for nucleic acids to bind with the PMBs. The permanent magnet will help in this mixing process before dragging the nucleic acid-bead complexes through the first oil valve into the washing chamber. The washing chamber contains solution slightly higher in ph to help remove unwanted proteins and salts carried along with the nucleic acids. Again, the permanent magnet will encourage mixing, and then move the nucleic acid-bead complex along to the elution chamber for elution. Here the ph level of the solution is significantly higher than the starting solution to reverse the binding process. In a more basic environment, the nucleic acids dissociate from the PMBs.

The analyzer may further comprise optical modules for excitation/detection and thermal modules for actuation/sensing. These modules may be controlled by a processing unit such as a computer or a microcontroller unit (MCU) to fully automate the sample-to-answer process on the disposable cartridge.

The permanent magnet provides full automation of the device to control the PMB movement within the cartridge. The PMBs can be bound and unbound from nucleic acids by adjusting the pH of the solution accordingly.

In one embodiment, the microfluidic cartridge comprises a cover layer, a microfluidic spacer layer, and a base layer. Alternatively, the cartridge can also be made using various plastics based manufacturing technique, including injection moulding, extrusion, blow molding, rotational molding, thermoforming, expanded bead foam molding and extruded foam molding, and 3D printing.

The microfluidic cartridge may further comprise valve chambers such as oil valve chambers located between the adjacent functional chambers such that a surface tension could be built up to separate the mixing of reagents in adjacent chambers.

The microfluidic cartridge may further comprises air traps and inlet ports.

The NAT device may be powered by a battery.

The present invention further provides methods of automated nucleic acid extraction and testing using the present nucleic acid testing device. The method may comprise the steps of providing a paramagnetic bead (PMB) assay bound to nucleic acids forming nucleic acid-bead complexes in the binding chamber, driving the nucleic acid-bead complexes into the washing chamber using the permanent magnet to remove unwanted proteins and salts carried along with the nucleic acids, moving the nucleic acid-bead complexes into the elution chamber using the permanent magnet for elution where the nucleic acids dissociate from the PMBs, and moving the PMBs away from the reaction chamber using the permanent magnet, leaving the nucleic acids in the reaction chamber for downstream analysis.

The platform could be used for all kinds of pathogen RNAs such as HIV, HPV, HBV, HCV RNAs. The platform could also be used for all kinds of pathogen DNAs. The sample may be blood, plasma, urine, saliva, virus transfer medium other common sample types.

### BRIEF DESCRIPTION OF THE DRAWINGS

FIG. 1A is a perspective view of a NAT-on-USB device according to an embodiment of the present intion (The enlarged inset shows an exploded view of the cartridge);

FIG. 1B is an exploded perspective view of the NAT device;

FIG. 1C is an assembled view of the inner portions of the NAT device with the cartridge in the operation position:

FIG. 1D is a top view of the NAT device, showing the electromagnetic array and the permanent magnet;

FIG. 2A is a perspective exploded view of an embodiment of the NAT device showing all internal components;

FIG. 2B is a cross-sectional view of the NAT device that demonstrates the geometry setup for the computer vision module and staging area;

FIG. 2C is a side view of the microfluidic cartridge to establish the physical relationship between the magnetic robot, printed circuit board (PCB), and paramagnetic beads (PMB) and a detailed view of the specific geometry of the EM PCB where coils are located on the top and bottom of the board.

FIG. 2D is a cross-sectional view of the device geometry with the cartridge sitting below the EM PCB and with the magnetic robot above it;

FIG. 3 shows an operation workflow of a HIV NAT-on-USB device;

FIG. 4A shows an example of the PCB design of the double-sided planar coil array;

FIG. 4B shows an operation diagram of the permanent magnet being moved from an initial position to a new adjacent position by turning on a specific coil (coil 2 in this case);

FIG. 4C shows calculated force on the permanent magnet as a function of the relative displacement of the permanent magnet to the coil center;

FIG. 4D shows a COMSOL simulation of the electromagnetic field generated by the planar coil;

FIG. 4E shows a sequence of the current pulse to move the permanent magnet from position 1 to position 3; in this example each pulse is 100 ms in duration;

FIG. 4F shows the corresponding permanent magnet position at each time spot during the pulsed operation;

FIG. 4G shows a Joule heating evaluation for the programmable electromagnetic pulse;

FIG. 4H shows the time course of the temperature measured on the planar coil surface at different operating frequencies corresponding to those in FIG. 4G;

FIG. 5 shows automated sample preparation and amplification on cartridges enabled by the EM pulsed actuation of charge-switchable magnetic beads;

FIG. 6A shows an exploded view of the PMMA layers that make up an embodiment of the microfluidic cartridge;

FIG. 6B shows a top view of the cartridge displaying the three-chamber structure separated by oil valves;

FIG. 6C is a schematic of magnetic robot motion and bead response;

FIG. 6D is a photo image of the cartridge with paramagnetic beads in each chamber;

FIG. 6E is a graphical representation of a Charge Switch assay;

FIG. 7A shows an original cartridge in Cartridge Durability Testing directly after fabrication and loading:

FIG. 7B shows a cartridge that were dropped from 3 ft and then imaged in Cartridge Durability Testing:

FIG. 7C shows a cartridge that were then dropped from 6 ft and then imaged in Cartridge Durability Testing:

FIG. 7D shows a cartridge that were sealed in plastic zipper bags and carried on-person during a daily commute, then imaged in Cartridge Durability Testing;

FIG. 8A is a schematic representation of the numerous electromagnet coils that are built into an embodiment of the electromagnet PCB;

FIG. 8B shows that magnet motion is achieved by turning on the next EM coil in the desired direction of motion; this can be done simultaneously in any of the four cardinal directions;

FIG. 8C shows a photo image of the EM PCB highlighting where the actual EM coils are located; in this example more than 50% of the PCB area is dedicated to the EM coil array;

FIG. 8D is a 2D planar representation of the magnetic field generated by current inside one EM coil, with a 3D coil simulation demonstrated through four 2D slices along the length of the coil;

FIG. 8E shows accuracy of the magnetic robot's motion vs the length of time of activation for the EM coils in both X and Y directions;

FIG. 8F shows temperature of the microfluidic cartridge over time when constant EM pulses are held at various frequencies;

FIG. 8G are thermal images of the PCB over time during an extraction protocol:

FIG. 9A shows power per pulse for varying pulse durations;

FIG. 9B are raw data plots of power for each recorded trial starting from 50 ms (top) to 800 ms (bottom) for X-Direction movement;

FIG. 9C are raw data plots of power for each recorded trial starting from 50 ms (top) to 800 ms (bottom) for X-Direction movement for Y-Direction movement;

FIG. 9D shows summarized total power consumption of the device when recorded over three separate trials;

FIGS. 10A-10C are thermal distribution and response images of each coil group for Y-Direction motion;

FIGS. 10D-10F are thermal distribution and response images of each coil group for X-Direction motion;

FIG. 10G shows cartridge temperature over the duration of an actual extraction procedure;

FIG. 11A is a block diagram explaining the logic flow through the camera vision algorithm for magnetic robot movement in an exemplary embodiment;

FIGS. 11B-11D are photo images of the EM PCB overlayed with the movement pathway of the magnetic robot for the numbered pathways;

FIG. 12A shows amplification curves for triplicate testing of synthetic RNA extraction from 50 uL plasma samples;

FIG. 12B shows input copy number vs Cq value from PCR analysis, limit of plasma extraction at 1000 copies;

FIG. 12C shows gel-electrophoresis panel of RNA input panel after PCR amplification;

FIG. 12D shows amplification curves of synthetic RNA extracted using silica PMBs;

FIG. 12E shows input copy number vs Cq value for silica PMBs;

FIG. 12F shows gel of PCR products after silica PMB extraction;

FIG. 13 is a table of cartridge and assay materials for an exemplary embodiment;

FIG. 14 is a table of ProMagBot device components for an exemplary embodiment;

FIG. 15 shows a RT-qPCR primer set targeting HIV-1;

FIG. 16 shows validation of the HIV-1 RT-LAMP assay with the quantitative panel; (A) primer set for HIV-1 subtype B RT-LAMP amplification;

FIG. 16B shows a RT-LAMP reaction setup for an exemplary embodiment;

FIG. 16C shows Real-time RT-LAMP amplification data with serially diluted HIV RNA standards for an exemplary embodiment (each concentration was repeated six times);

FIG. 16D shows time to positive value at different HIV-1 RNA concentrations for an exemplary embodiment;

FIG. 16E shows a summary of the hit rate for an exemplary embodiment;

FIG. 17A shows RT-LAMP amplification curves with serially diluted HIV RNA standards with spiked whole blood mock sample for an exemplary embodiment;

FIG. 17B shows a photo image of RT-LAMP products observed in the reaction tubes under a UV light for an exemplary embodiment;

FIG. 17C shows a gel image of RT-LAMP products analyzed by agarose gel electrophoresis for an exemplary embodiment;

FIG. 17D shows the extracted hit rate at various RNA concentrations to establish the assay LoD, which is determined to be 214 copies/mL at the 95% confidence level, for an exemplary embodiment;

FIG. 17E shows time to positive value at different HIV-1 RNA concentrations in whole blood for an exemplary embodiment;

FIG. 18A shows a photo image showing an embodiment with multiple analyzers being used simultaneously through a single USB hub;

FIG. 18B shows real-time RT-LAMP data in the intra-device test with a serially diluted mock blood sample for an exemplary embodiment; each concentration was tested in triplicates;

FIG. 18C shows extracted time to positive value for the intra-device test;

FIG. 18D shows the scattering plot of the time to positive value between two devices;

FIG. 18E shows the end-point fluorescence values for a total of 104 whole blood samples (52 negatives and 52 positives) tested with four different analyzers;

FIG. 19A shows a photo image of an exemplary optical module;

FIG. 19B shows optical responsibility of the optical sensor as well as the calcein emission profile for an exemplary embodiment;

FIG. 19C shows the circuit schematic diagram of the excitation and emission sensing module for an exemplary embodiment;

FIG. 19D shows characterization of the optical sensor with calcein;

FIG. 20A shows an embodiment of the thermal module that is composed of a power resistor as a heating source, a thermistor as a temperature feedback sensor, and a CNC aluminum plate;

FIG. 20B shows a photo image of each component in the heating module (left) and the assembled heating module (right) for an exemplary embodiment;

FIG. 20C shows a photo image of the thermal module assembled on the analyzer (top view) for an exemplary embodiment;

FIG. 20D shows a flow chart of the thermal control algorithm for an exemplary embodiment;

FIG. 20E shows that an exemplary embodiment of the thermal module can reach the required 60° C. within 1.5 minutes, and the root mean square (RMS) value of the temperature after stabilization is 0.53° C., which can meet the temperature requirements of LAMP detection;

FIG. 21 shows a schematic diagram of the device software and hardware for an exemplary embodiment;

FIG. 22 shows the real-time RT-LAMP curves for the inter-device test (4 devices) at different HIV-1 RNA concentrations in whole blood for an exemplary embodiment:

FIG. 23 shows a bill of materials of and exemplary embodiment of the analyzer; and

FIG. 24 shows microfluidic cartridge and reagent cost per test for an exemplary embodiment.

### DETAILED DESCRIPTION OF THE INVENTION

The embodiments of the present invention provide an automated nuclei acid testing (NAT) device. The NAT device simplifies the process of sample preparation by automating nucleic acid extraction.

The NAT device may include a housing and a microfluidic reagent cartridge housed in the housing. The microfluidic reagent cartridge can be inserted into and removed from the housing. The cartridge includes a binding chamber, a washing chamber and a reaction chamber. Each of these functional chambers are separated by an oil valve chamber. The cartridge may be disposable and be preloaded with all reagents needed for operating a paramagnetic bead (PMB) assay. For actuating the nucleic acid-bearing magnetic beads on the cartridge, a magnetic robot may be used. The magnetic robot includes a double-sided planar electromagnetic coil array on a printed circuit board (PCB) paired with a permanent magnet. This PCB coil can be programmed to generate a localized pulsed electromagnetic field for actuating the permanent magnet that further controls the paramagnetic beads in the cartridge. The paramagnetic beads may be charge-switchable paramagnetic beads. The bead's surface charge is dependent on the surrounding reagents' pH value, i.e., the surface charge can be switched from positive to negative when pH is below or above 7. The electromagnetic coils and permanent magnet may be located above the cartridge or below the cartridge. The electromagnetic coil array can be a one-dimensional (1D) array or a 2-dimensional (2D) array. For a 2D array, the electromagnet design contains groups and blocks of coils that are actuated in series. The permanent magnet provides full automation of the device to control the PMB movement within the cartridge. The PMBs can be bound and unbound from nucleic acids by adjusting the pH of the solution accordingly.

The NAT device may be further integrated with optical modules for excitation/detection and thermal modules for actuation/sensing. These modules may be controlled by a processing unit such as a computer or a microcontroller unit (MCU) to fully automate the sample-to-answer process on the disposable cartridge. Optical, thermal, and electromagnetic array subsystems are seamlessly integrated to perform streamlined nucleic acid testing. The NAT device can be designed to be USB-interfaced for data connection. Multiple NAT devices can be connected concurrently through a single USB hub to a computer processing unit such as a PC. On the PC side, a graphic user interface (GUI) is designed to automatically recognize and administrate the analyzer inserted through the USB port. The final positive/negative results will be displayed on GUI to the end-user.

The NAT device can be powered by a rechargeable lithium polymer (LiPo) battery and certain embodiments consume 1.22 Wh per extraction run. Users collect a testing sample, mix it with the lysis and charge switchable magnetic bead buffer, and insert the solution into the microfluidic cartridge. The microfluidic cartridge may be extremely robust and contain all reagents needed for operating a paramagnetic bead (PMB) assay. Charge switchable PMBs offer an extraction assay that is friendlier to handle by laypersons since there is no need for guanidium, ethanol, or isopropanol. The NAT device and protocol are also compatible with modified versions of silica coated PMB extraction kits.

The NAT device can be used for nucleic acid testing such as HIV RNA testing. Here is an example of using the present NAT device for the HIV RNA testing. In the first step, the negatively charged RNAs in the lysate bind to the positively charged magnetic beads at pH 5 in the binding chamber. During the binding process, the permanent magnetic under or above the cartridge is actuated back and forth at a frequency to ensure thorough mixing. In the second step, the RNA binding beads were transferred to the washing chamber (buffered at pH 7) by the EM array. The beads were horizontally agitated by the programmed EM sequence. In the third step, the washed beads are transferred to the reaction chamber with the master mix buffered at pH 8.8. The RNAs are directly eluted to the master mix due to the positive charge on the magnetic bead surface. After elution, these magnetic beads were moved away from the reaction chamber (step 4) before the eluted solution is used for downstream analysis. The entire sample preparation could be completed in less than 15 minutes with minimum user interaction.

Some embodiments of the NAT device can be utilized in areas of low resources for nucleic acid extraction in under 20 minutes. A trained professional is not necessary to operate the NAT device so it can also be used for self-test at home for privacy.

In one embodiment for HIV self-testing, the device consists of a microfluidic reagent cartridge and an ultra-compact NAT-on-USB analyzer. The device can work with a reduced whole blood volume of 100 μL (readily available with finger-pick method) as compared to traditional methods using ˜10 ml of venous blood (LabCorp). The test requires simple steps from the user to drop the finger-prick blood sample into a collection tube with lysis buffer and load the lysate onto the microfluidic cartridge, and the testing result can be easily read out on a custom-built graphical user interface (GUI). The microfluidic cartridge can automatically handle the complexity of sample preparation, purification, and real-time reverse-transcription Loop-mediated Isothermal Amplification (RT-LAMP). The automation is facilitated by the programmable electromagnetic (EM) pulse method of the present invention. The highly portable analyzer is USB interfaced and integrates cooperating subsystems (electronic, optical and mechanical) into an ultra-compact form factor. With the embodiment of the present invention, the HIV self-testing could be performed as simply as a home blood glucose test. The rapid, low-cost, easy-to-use HIV NAT-on-USB would be particularly useful for the high-risk populations seeking private, highly sensitive self-testing at home.

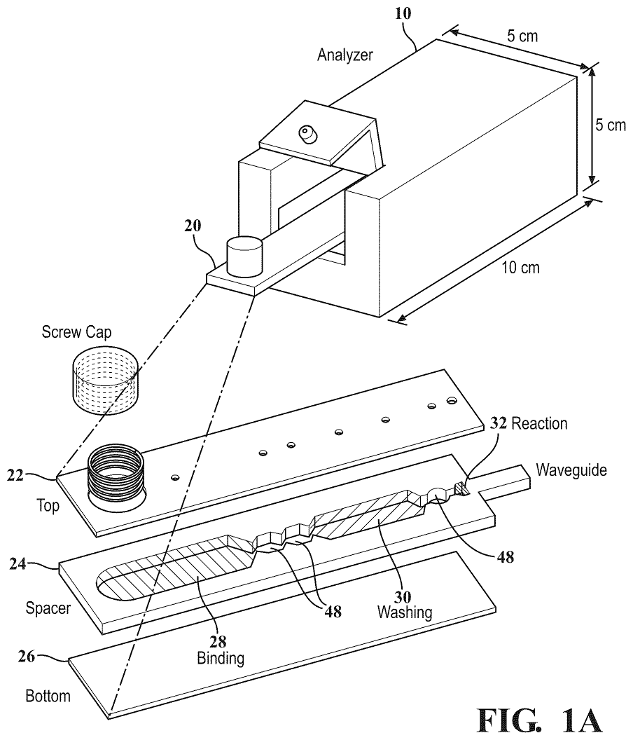

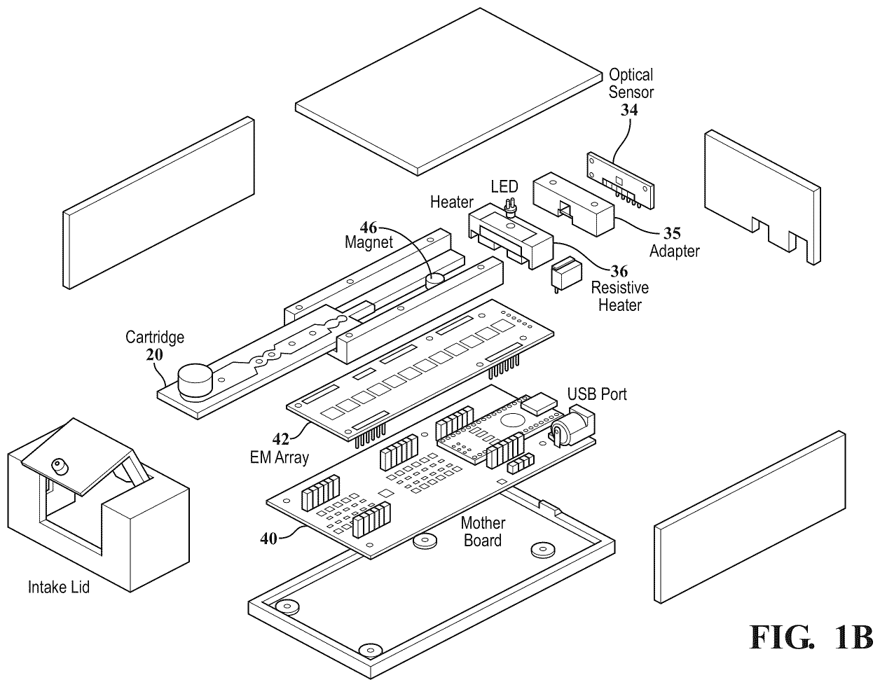

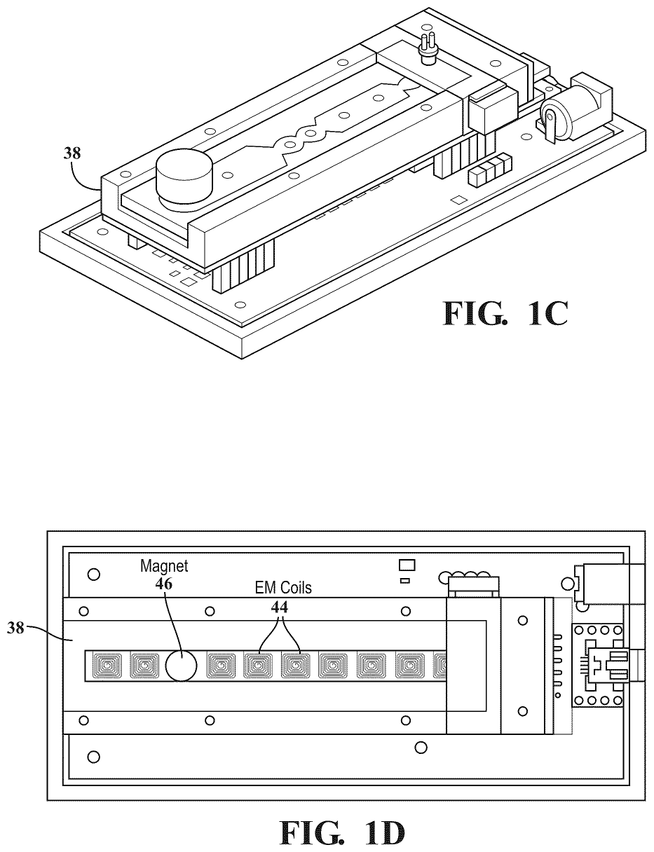

FIG. 1A illustrates an embodiment of a NAT-on-USB device 100 for HIV self-testing. The device 100 includes a highly portable palm-sized analyzer 10 (footprint of 10×5×5 cm³, weighing 170 g) and a ready-to-use, disposable reagent cartridge 20. The inset of FIG. 1A shows an exploded view of the cartridge design with an overall dimension of 9 cm (1)×1.5 cm (w)×0.58 cm (h). The cartridge 20 consists of three-patterned polymethyl methacrylate (PMMA) layers, i.e., top 22, spacer 24 and bottom 26, laminated with an adhesive solvent. Alternatively, it may be formed in other ways. The assembled cartridge has a binding chamber 28 (800 μL), a washing chamber 30 (450 μL), and a reaction chamber 32 (25 μL). Each of these functional chambers are separated by an oil valve chamber 48. Preferably, the angle between the adjacent walls of the chambers are equal to or greater than 270 degree. For example, the angle between the adjacent walls of the binding chamber and the valve chamber is at least 270 degree. Reagents are preloaded to the cartridge before use.

FIGS. 1B and 1C show an exploded view and an assembled (without top casing) view of the analyzer 10, respectively. The USB-interfaced analyzer integrates the optical modules for excitation/detection, thermal modules for actuation/sensing, and mechanical modules 38 including a printed circuit board (PCB) 42 coil electromagnet driver. The optical module includes an optical sensor 34, a LED, an adapter 35. The adapter is to mechanically secure the optical sensor and auto-align the optical sensor to the reaction chamber. The thermal module comprises of a resistive heater 36, a temperature sensor for feedback control as well as an aluminum based block. The temperature sensor is embedded inside the aluminum block but electrically isolated. These modules are controlled by a microcontroller unit (MCU) 40 to fully automate the sample-to-answer process on the disposable cartridge.

For actuating the nucleic acid-bearing magnetic beads on the cartridge, a double-sided planar coil array 44 on a printed circuit board (PCB) 42 is used. This PCB coil 44 can be programmed to generate a localized electromagnetic (EM) field for actuating a permanent magnet 46, shown in FIG. 1D.

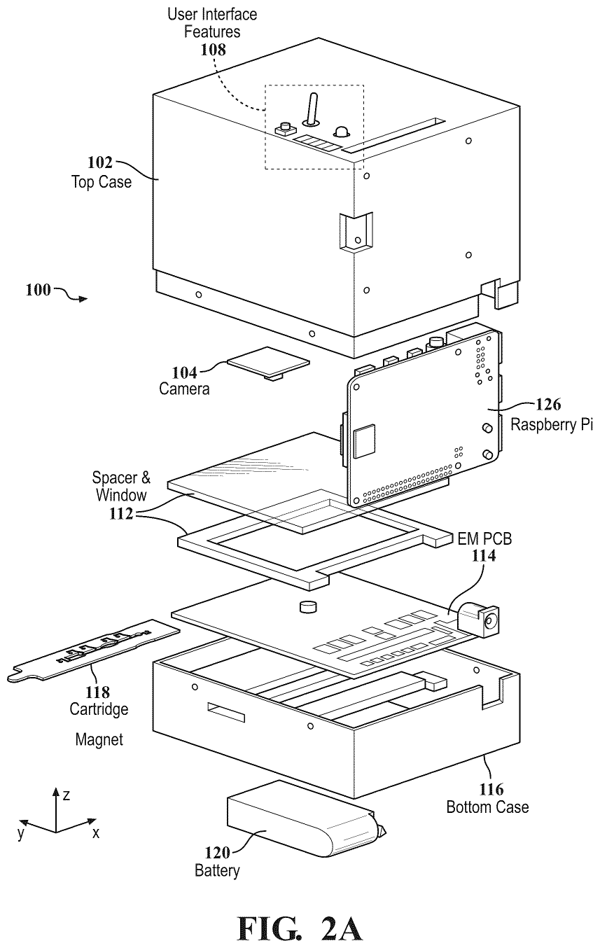

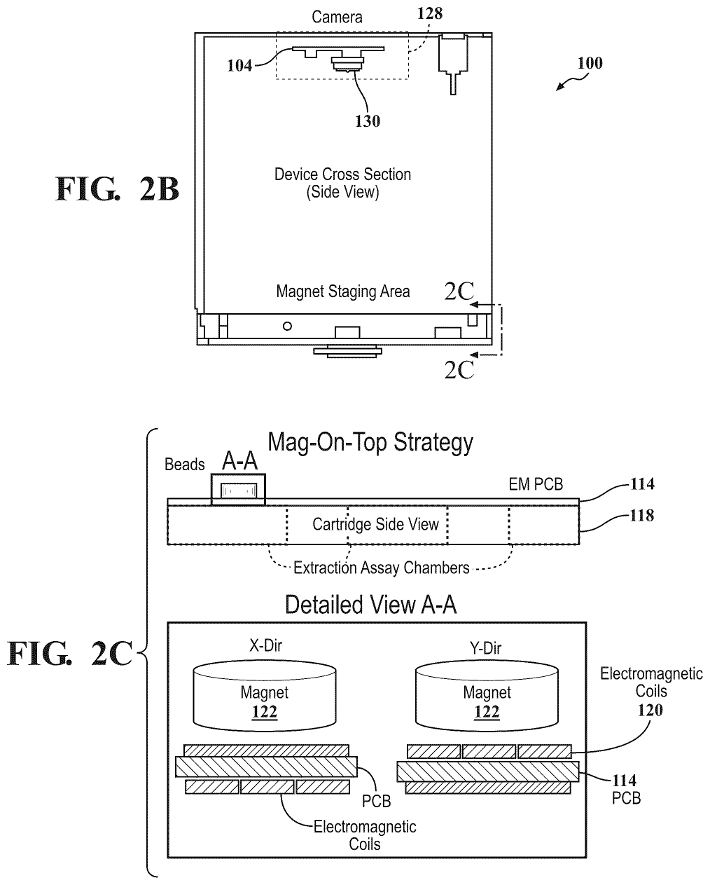

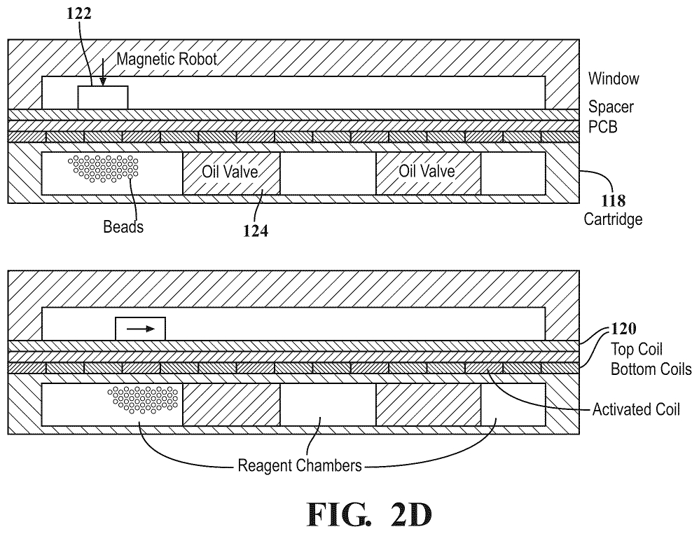

As seen in FIG. 2A, the automated NAT device 100 is composed of multiple parts: (generally from top to bottom) a top casing 102, a camera 104, a light guide 130, user interface features 108, a processor 126 (in this example a Raspberry Pi 4), a spacer and viewing window 112, an electromagnetic PCB 114, a bottom casing 116, a microfluidic cartridge 118, and thae compact battery 120. The device can be divided into three subcategories: a computer vision system 128 in FIG. 2B, a microfluidic cartridge 118, and an electromagnetic PCB 114 paired with a permanent magnet 122 shown in FIG. 2C.

FIG. 2D shows a clearer cross-sectional view of an embodiment of the microfluidic cartridge 118 which contains all the necessary reagents for the charge switchable nucleic acid extraction assay, each separated by oil valve chambers 124. Located above the microfluidic cartridge 118, the electromagnetic coils 120 and permanent magnet 122 allow the NAT device to employ a magnet-on-top (Mag-On-Top) approach. This enables the device to include chamber mixing through passive forces. Gravity precipitates the beads when not in the presence of the magnet and improves the mixing in each chamber. Computer-aided tracking of the permanent magnet within the electromagnetic coil array provides increased rigidity for the magnet pathway and allows for redundancies against improper motion, currently not seen in other POC devices.

The NAT device simplifies the process of sample preparation by automating nucleic acid extraction. The device uses a LiPo battery for ultra-portability and is handheld in size. The cartridge is extremely robust and able to withstand drops from 3 ft and 6 ft without reagent disruption. With the magnetic robot on top of the cartridge, the Mag-On-Top geometry is introduced to increase the mixing capabilities.

### Exemplary Workflow

The following describes an exemplary workflow for an embodiment of the invention, but is only an example and is not limiting on other embodiments.

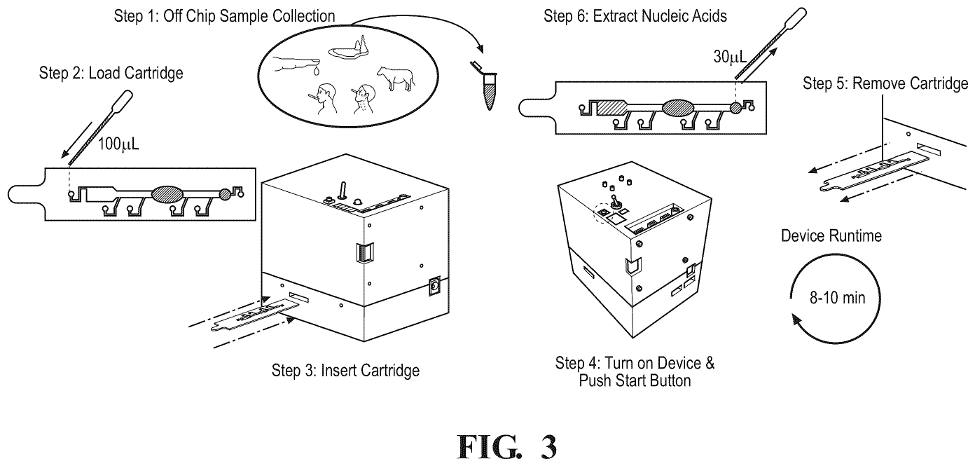

User interaction with the device follows the steps illustrated in FIG. 3. First, the user collects the sample off chip and transfers it to a collection tube. Using a one hundred μL pipette, the user transfers the lysate mixture into the binding chamber of the microfluidic cartridge and inserts the cartridge into the analysis device. Then, the user can power on the device. Once on, the device will initiate a bootup sequence, and when ready, indication LEDs will flash. The device will run for 8-10 minutes. The device will signal when the extraction is complete using the indicator lights. Then, the user removes the cartridge from the device in the opposite direction as insertion. Lastly, the eluted nucleic acids can be removed from the cartridge. The extracted elution and concentrated nucleic acids can then be used for further downstream analysis or frozen for long-term storage.

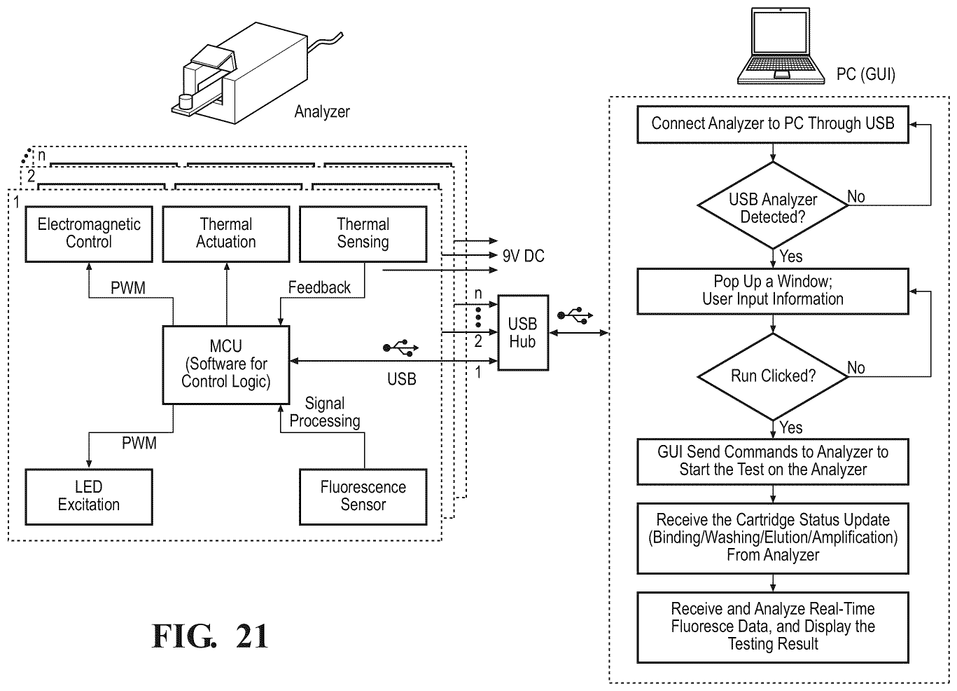

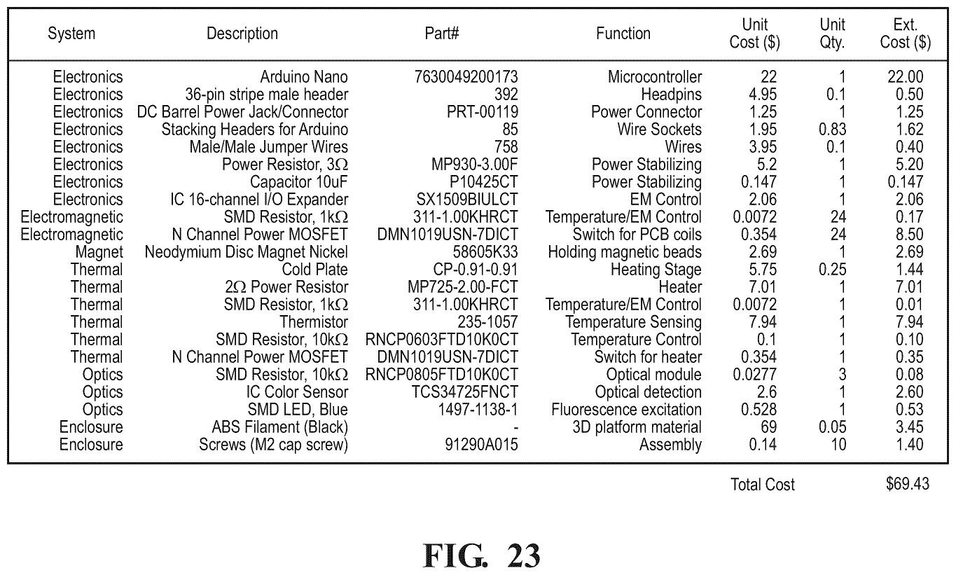

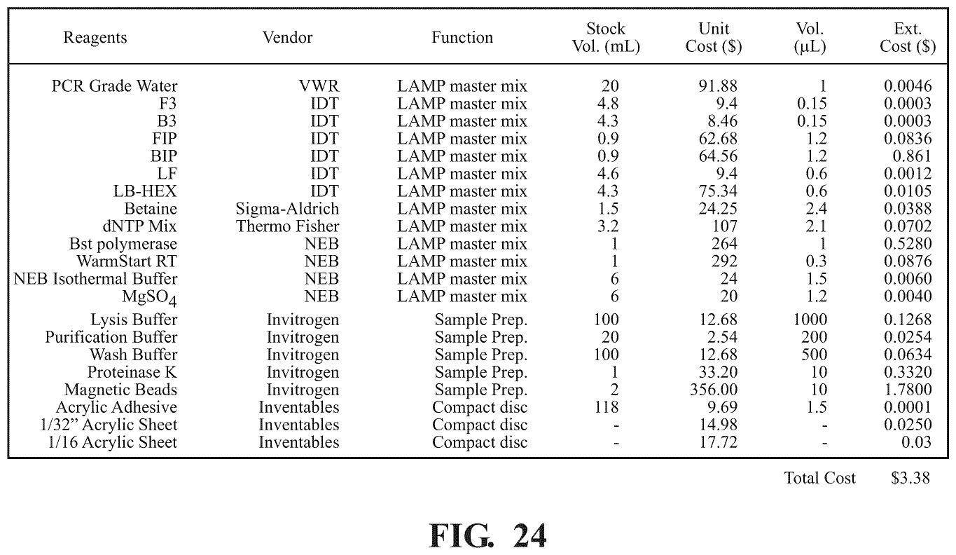

For the HIV NAT-on-USB, the user would self-collect ˜100 μL of finger-prick blood using an exact volume transfer pipet and drop it into a collecting tube pre-filled with 800 μL lysis buffer. 200 μL binding buffer, and 15 μl, charge switchable magnetic beads. After the blood is collected into the lysis tube, the user can shake the tube to promote the mixing and binding. After 1 min, the lysate is loaded onto the binding chamber of the microfluidic cartridge through the extruded inlet, which can be completely sealed with a screw cap by hand tightening. The sealed cartridge is then inserted along a sliding rail into the analyzer through a hinged intake lid. After closing the lid, the analyzer is connected to a personal computer (PC) through a USB port. A customized PC graphical user interface (GUI) was developed for interfacing with the analyzer and interpreting the data in a user-friendly way. The test will be automatically recognized and administrated by the GUI. The GUI can automatically detect a new analyzer connection, request user information, initiate the nucleic acid test, and report the ‘yes/no’ qualitative result. FIG. 21 shows a schematic diagram of an exemplary embodiment of the device software and hardware. On the analyzer side, the optical, thermal, and electromagnetic modules are seamlessly integrated and controlled by an MCU for automated nucleic acid testing. The analyzer is designed to be USB-interfaced for data connection. Multiple analyzers can be used concurrently through a single USB hub. On the PC side, a graphic user interface (GUI) is designed to automatically recognize and administrate the analyzer inserted through the USB port. The device status, real-time amplification data, and the final positive/negative results will be displayed on the GUI to the end-user. The final positive/negative results will be displayed at the end of the automated process. The USB-interfaced analyzers can be used in a plug-and-play (PnP) fashion. In addition, multiple USB-interfaced analyzers can be simultaneously and independently connected to a PC through a USB hub for enhanced throughput, if needed. In an example, the material cost per test is $3.30 per reagent cartridge and $69.43 per analyzer (Sec Tables in FIGS. 23 and 24). The microfluidic cartridge is an enclosed system after the sample is loaded. It is disposable after each test. Therefore, cross-contamination between tests is not a concern. The overall HIV NAT-on-USB workflow requires minimal user intervention and is simple enough for the laypersons to perform HIV self-testing.

### Planar Coil Example

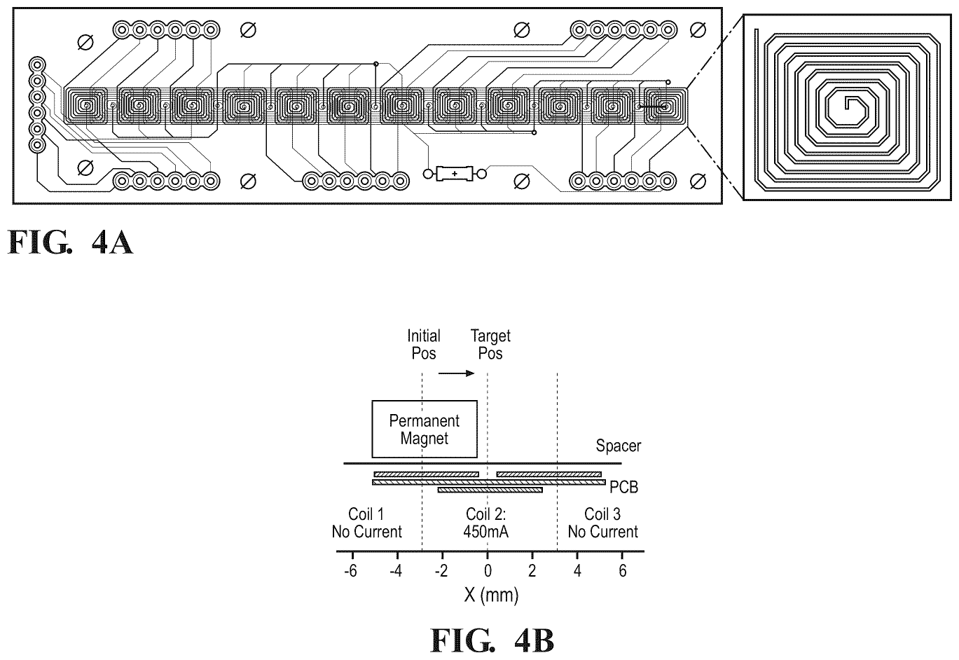

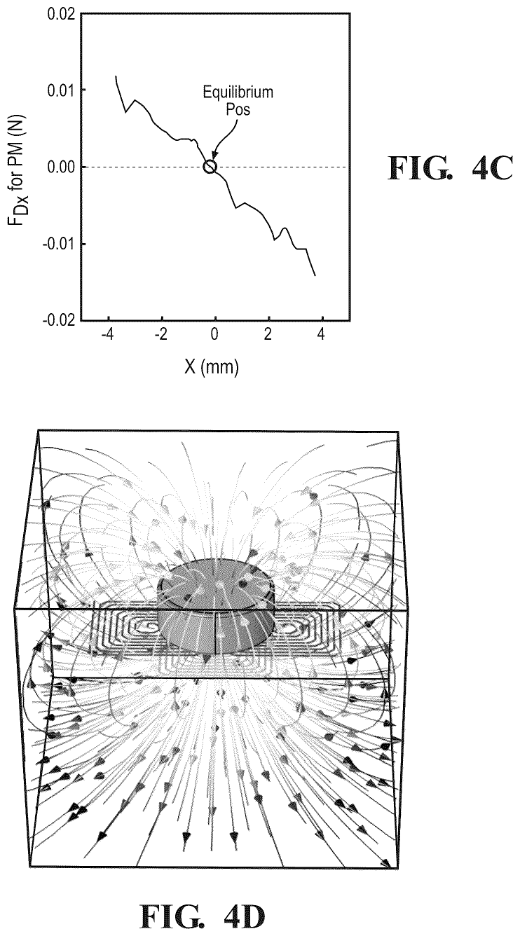

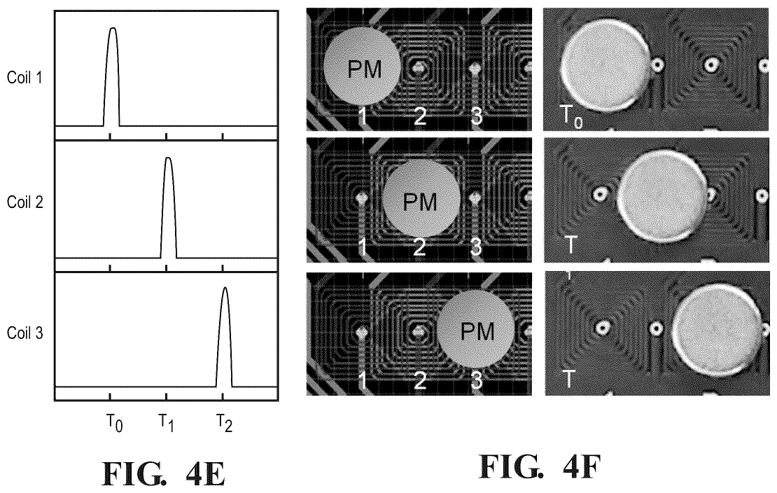

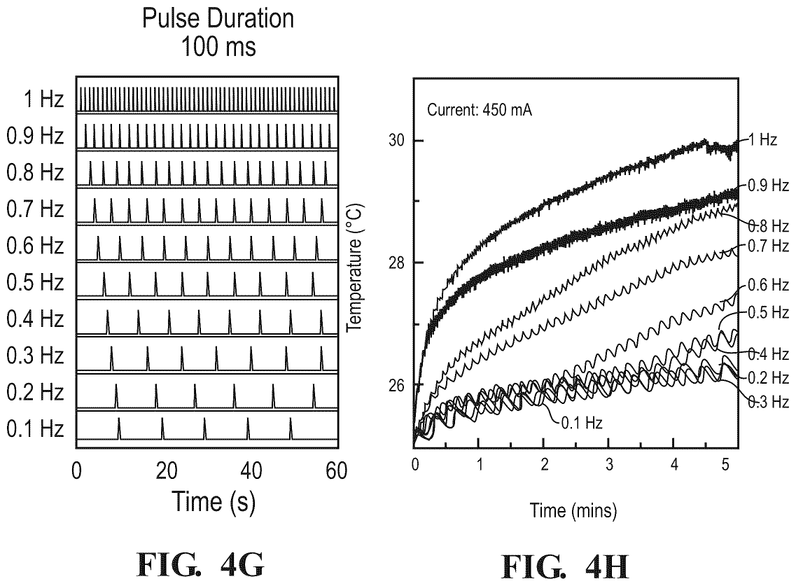

FIG. 4A shows an embodiment of a programmable electromagnetic method using double-sided planar coil array to completely remove the bulky moving parts for actuating the magnetic beads in the NAT-on-USB device. The planar coil is designed into two layers in a single PCB. There are 12 coils on the top layer and 11 coils on the bottom layer (1×23 array). The permanent magnet can be programmed to the center of any of these 23 coils. This is because a deviation from the ‘ON’ coil will result in a restoring force to bring the permanent magnet to its equilibrium position (center of the ‘ON’ coil), as shown in FIGS. 4B-4D. The planar coil is designed as two layers in a single PCB with a vertical distance of 0.78 mm. Each rectangular coil has a winding width of 170 μm, a spiral pitch of 170 μm, a thickness of 35 μm, and nine turns (enlarged inset). The coils on the top and bottom layers are offset by 3.6 mm horizontally, yielding an effective motion step of 3.6 mm.

The programmable pulsed EM field is used to actuate a permanent magnet that further controls the magnetic beads on the cartridge, shown in FIGS. 4E and 4F. Since the permanent magnet itself has a substantial susceptibility, a small electromagnet field (i.e., a reduced power consumption) is sufficient to drive its motion. In addition, the actuation of the permanent magnet only requires 100 ms of ‘ON’ time on the desired coil. With this pulsed operation, a minimum of 450 mA is sufficient to actuate the permanent magnet in the device. FIG. 4G shows Joule heating evaluation for the programmable electromagnetic pulse. The current pulse waveform with 0.1 Hz to 1 Hz operating frequencies. The duration of each pulse is fixed at 100 ms. The temperature generated by the Joule heating was found to be operation frequency-dependent. FIG. 4H shows the time course of the temperature measured on the planar coil surface at different operating frequencies corresponding to these in FIG. 4G. The current pulse amplitude is 450 mA. With 0.1 to 1 Hz operation frequencies, the measured temperature does not exceed 30° C. for 5 min of operation, indicated in FIGS. 4G and 4H, suggesting that the reagents and assays in the cartridge would not be affected by the electromagnetic actuation, alleviating the overheating problems in previous methods (Chiou et al. 2013).

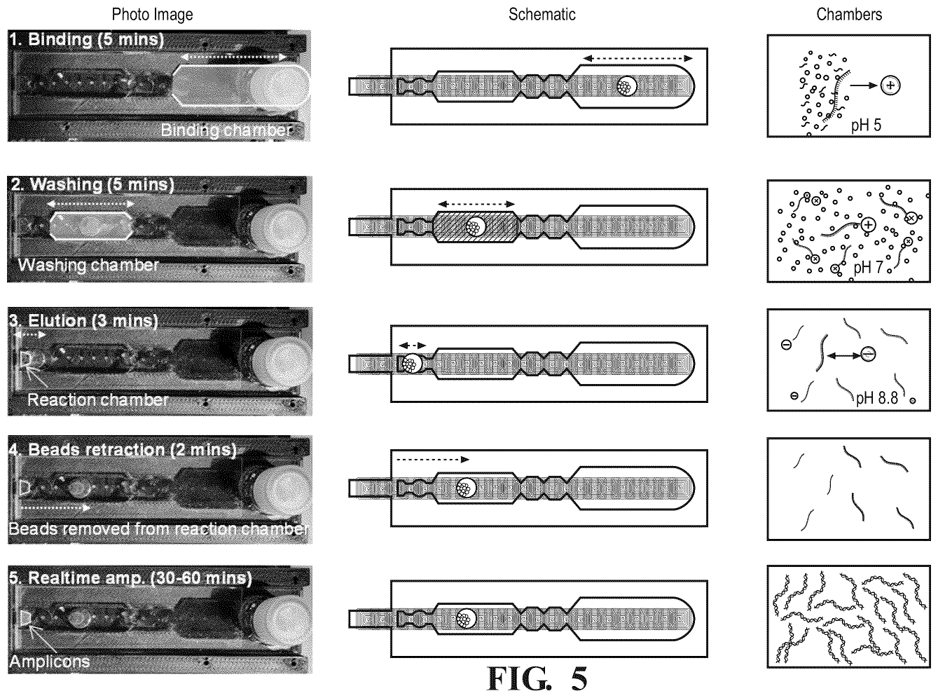

FIG. 5 illustrates the automated sample preparation and amplification on cartridges enabled by the EM actuation of charge-switchable magnetic beads. The left, middle and right panels show the photo image of the actual device, the schematic of the relative position of the cartridge chambers to the EM driven magnet, and the schematic interactions of molecules with magnetic beads, respectively. In the first step, the negatively charged RNAs in the lysate bind to the positively charged magnetic beads at pH 5 in the binding chamber. During the binding process, the permanent magnetic under the cartridge was actuated back and forth at a frequency of 1/3 Hz to ensure thorough mixing. In the second step, the RNA binding beads are transferred to the washing chamber (buffered at pH 7) by the EM array. The beads are horizontally agitated by the programmed EM sequence at 1/3 Hz. In the third step, the washed beads are transferred to the reaction chamber with the master mix buffered at pH 8.8.

The bead's surface charge is dependent on the surrounding reagents' pH value, i.e., the surface charge can be switched from positive to negative when pH is below or above 7. RNAs or DNAs would remain negatively charged in a large range of pH values. This is illustrated in FIG. 5 right column.

The RNAs are directly eluted to the master mix due to the negative charge on the magnetic bead surface. After elution, these magnetic beads are moved away from the reaction chamber (step 4) before starting the RT-LAMP reaction (step 5). The entire sample preparation could be completed in less than 15 minutes with minimum user interaction.

### Microfluidic Cartridge Design Example

The following describes an exemplary microfluidic cartridge design for an embodiment of the invention, but is only an example and is not limiting on other embodiments.

It was found through channel testing that thicknesses smaller than 2 mm were difficult for the bead aggregate to travel through due to adhesion to the channel sidewalls. It should be able to work in the range of 1-10 mm, although it is found that 2 mm is an optimized value Thus, a 2 mm channel thickness is chosen for these exemplary cartridges.

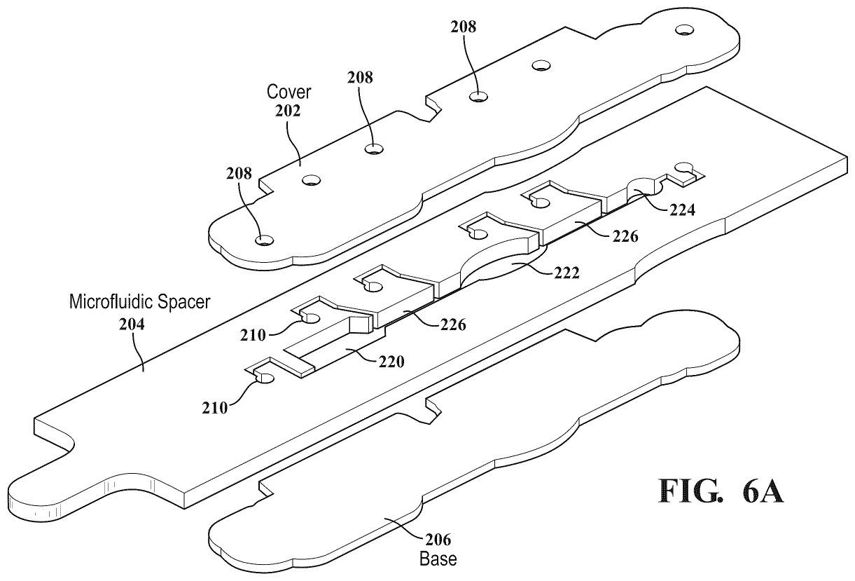

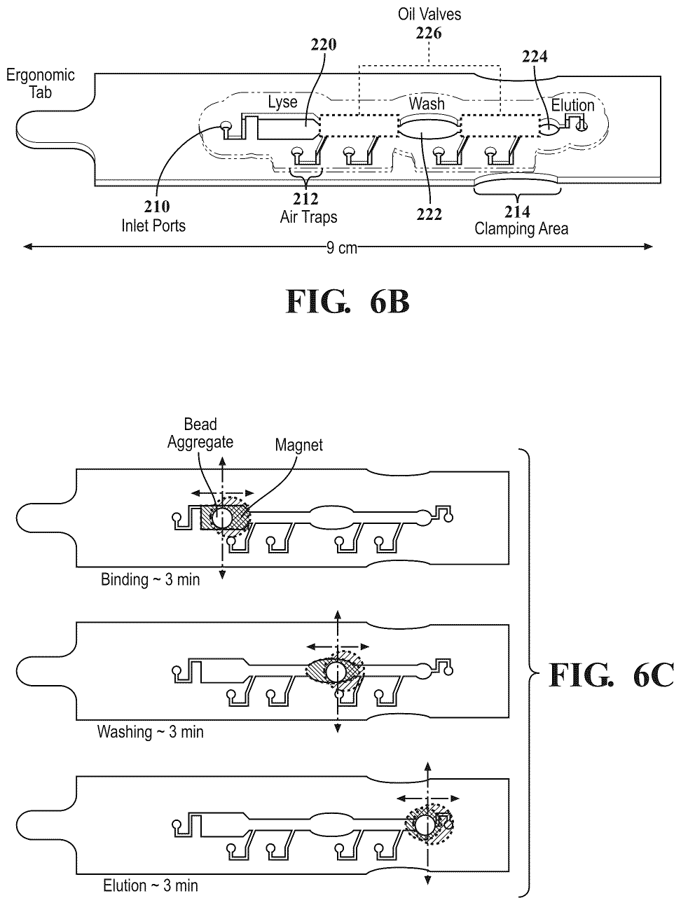

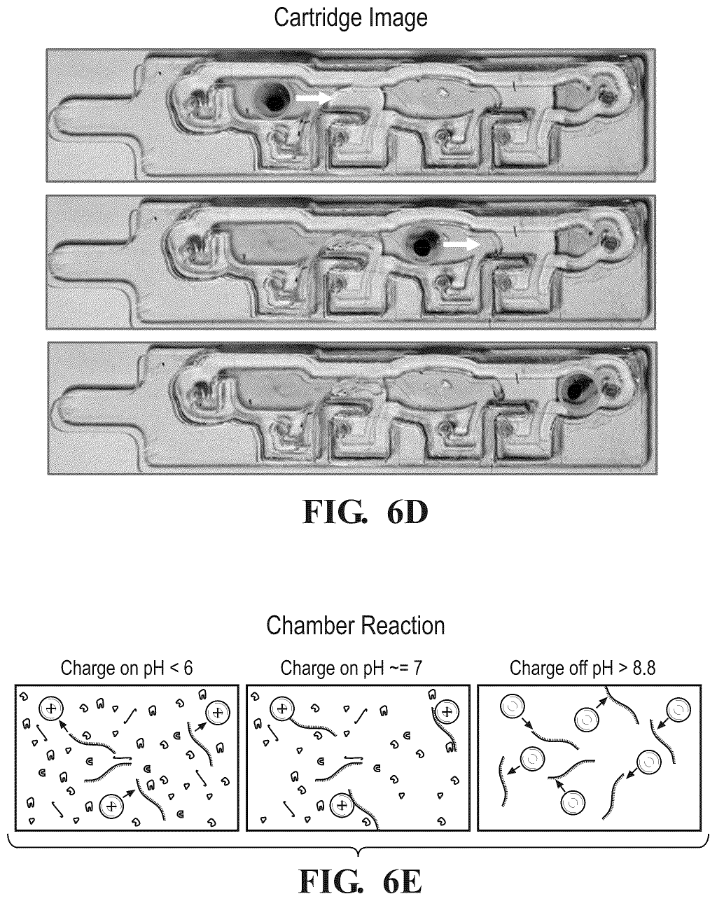

For the NAT system, a three-layered microfluidic cartridge is utilized to separate, contain, and encompass the extraction solutions. As seen in FIG. 6A, the microfluidic cartridge is composed of a cover layer 202 that contains inlet ports 208 for the liquid solutions. The microfluidic spacer 204 sits in the middle of the cartridge and creates the channel necessary for PMB movement from one solution buffer to another. The microfluidic spacer layer 204 includes a binding chamber 220, a washing chamber 222, an elution chamber 224 and oil valve chambers 226 between different chambers. Last, the bottom of the cartridge is sealed with a base layer 206 that squares off the cartridge and creates an entirely enclosed microfluidic channel. The chambers may be rectangular, square, round or the combination of them. The dimensions of the binding chamber, washing chamber, elution chamber may vary from 1 mm to 100 mm. The valve chambers could vary from 1-10 mm.

The cartridge can also be made using various plastics based manufacturing technique, including injection moulding, extrusion, blow molding, rotational molding, thermoforming, expanded bead foam molding and extruded foam molding, and 3D printing.

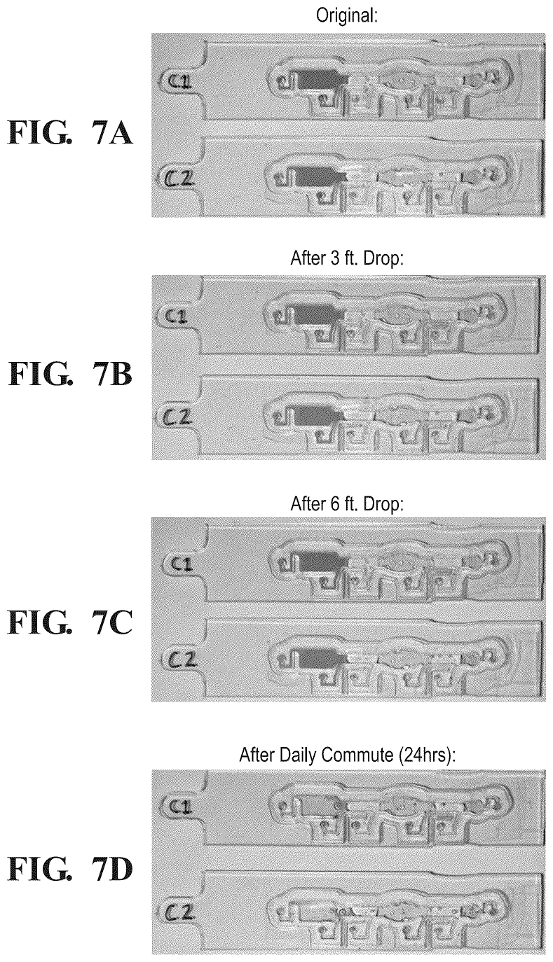

A more detailed view of the inside of the cartridge can be seen in FIG. 6B. Special air traps 212 and insertion ports 210 are located away from the main channel to prevent excess air bubbles. A clamping structure 214 is designed on the cartridge such that when the cartridge is inserted, it will be automatically clamped, similar to those used in SD card for computers. The clamping structure 214 allows for smooth insertion into the housing of the NAT device. The individual lysis, wash, and elution buffer chambers are labeled accordingly. Each chamber has two ports for air and solution. This helps to prevent air bubble accumulation when loading the cartridge. Oil valves are smaller chambers between adjacent chambers such that a surface tension could be built up to separate the mixing of reagents in adjacent chambers. Oil valves provide a great separation method for magneto fluidics and microfluidic cartridges. They improve the stability of the cartridge, minimize carry over volume, and do not inhibit any downstream detection methods. In FIGS. 7A-7D, we qualitatively tested the strength of our cartridge's oil valves. We tested the cartridges' ability to withstand drops of 3 ft and 6 ft. No valve or reagent disruption was seen when compared to the original state, but we also tested the ability of the cartridge to withstand “normal” travel in a 24-hour period. The cartridge was stored in a plastic bag and carried in a backpack to simulate an average commute. Here the cartridge reagents remained intact, but air bubbles could be seen inside the cartridge. The introduction of air bubbles signifies that the cartridge does not remain airtight, which can be detrimental to oil valve integrity and cartridge stability. However, we envision that improved manufacturing techniques will demonstrate better shelf lifetimes with more airtight cartridges.

FIGS. 6C-6E showcase three separate views of the paramagnetic beads as they are moved along through the cartridge. The paramagnetic beads can be bound and unbound from nucleic acids by adjusting the pH of the solution accordingly. This system's cartridge is designed so that the PMB assay can be bound to nucleic acids in the lysis chamber. In part one, the lysis buffer adds detergents and surfactants to the sample to break down cells, bacteria, and viral particles (FIG. 6C-6E left end). In part two, the binding buffer decreases the pH of the solution to a more ideal condition for nucleic acids to bind with the PMBs. The permanent magnet will help in this mixing process before dragging the nucleic acid-bead complexes through the first oil valve into the washing chamber. Second, the washing chamber contains solution slightly higher in pH to help remove unwanted proteins and salts carried along with the nucleic acids (FIG. 6C-6E middle). Again, the permanent magnet will encourage mixing, and then move the nucleic acid-bead complex along to the last of three chambers for elution. Here the pH level of the solution is significantly higher than the starting solution to reverse the binding process. In a more basic environment, the nucleic acids dissociate from the PMBs. After thorough mixing, the PMBs can be removed by magnet, leaving the nucleic acids in chamber three (FIG. 6C-6E right end). The remaining solution contains concentrated nucleic acids to be extracted by the user for downstream analysis.

Charge switchable extraction assays greatly reduce the chemical and professional requirements associated with sample preparation. These kits remove the need for toxic chemicals while still enabling nucleic acid extraction with magneto-microfluidics. The use of oil separated chambers creates a cartridge that is extremely robust and easy to use while still allowing bead manipulation. Therefore, the ProMagBot cartridge introduces an overly robust cartridge that remains functional with magnetic methods.

### Exemplary Electromagnet PCB Design

The following describes an exemplary electromagnet PCB design for an embodiment of the invention, but is only an example and is not limiting on other embodiments.

This embodiment introduces a battery-powered extraction device capable of 2-dimensional (2D) movement. The electromagnet design contains Groups and Blocks of coils that are actuated in series. First, this creates step wise coils that can be used sequentially to move a magnet in one direction or another. Second, this significantly reduces the complexity of the device's circuitry by eliminating the need for a multiplexer commonly required for addressable coils. Third, stepwise motion allows the device to operate in pulses that can minimize energy consumption. The device is capable of 4 directions of motion (±x,±y) by using six transistor switches.

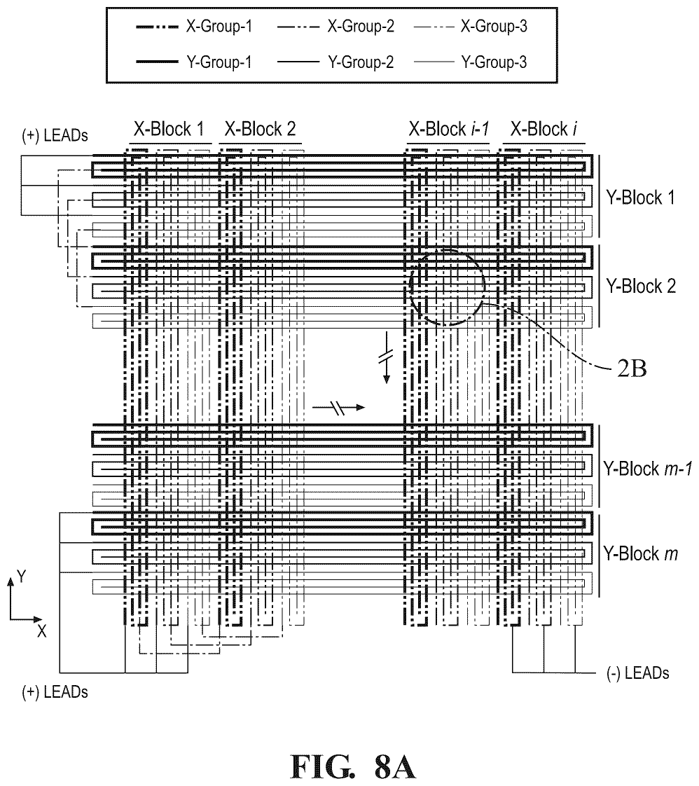

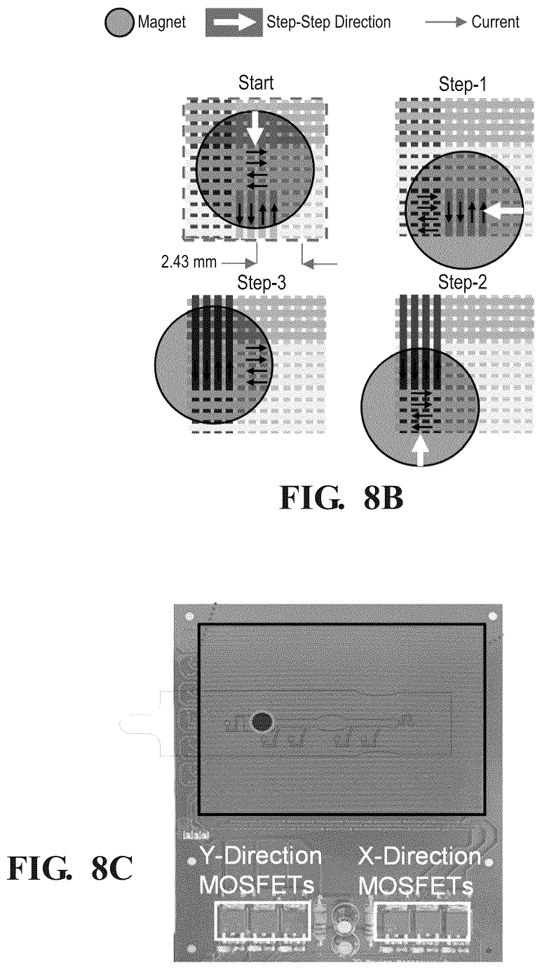

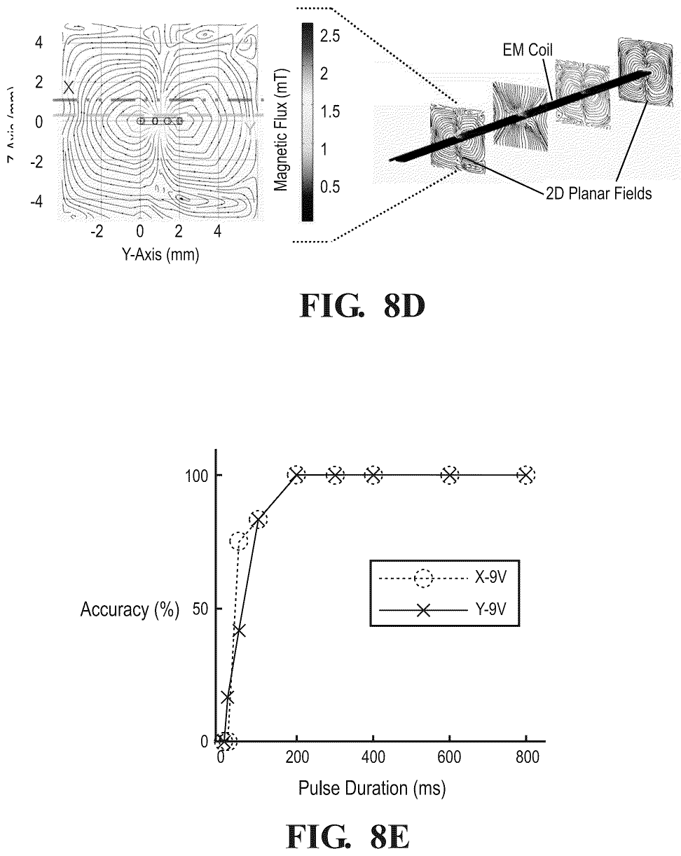

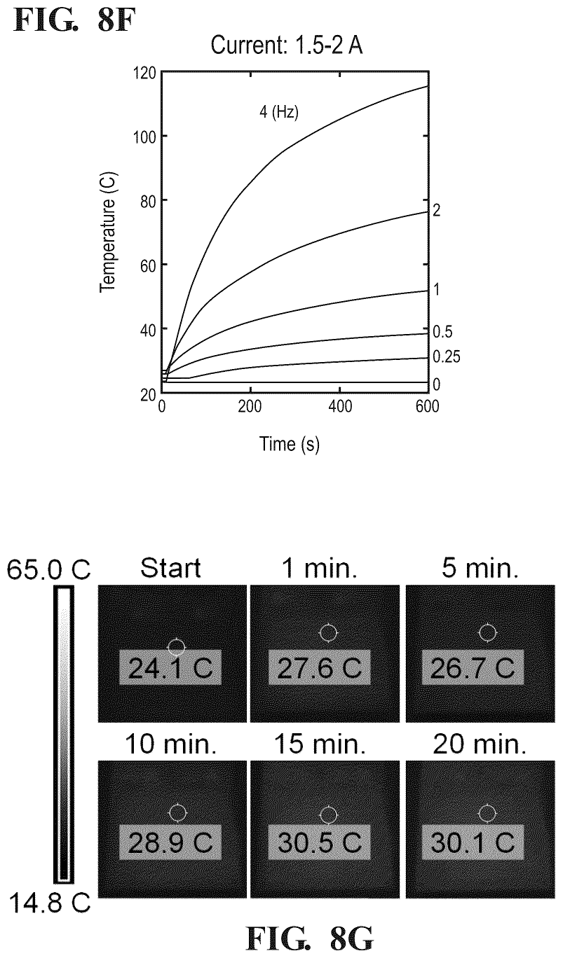

In FIG. 8A, these coils are defined by 21 different blocks, with each block holding three Groups. Coils are activated by Group designation and are interconnected across Blocks. Therefore, all coils of the same Group in all Blocks are turned on/off together. The coil layout is visualized for both the X and Y direction. For either direction, a Block contains three separate coils that each belong to a unique Group. Blocks are repeated 21 times in both X and Y, while individual Groups remain electrically connected. Therefore, when any one Group is activated, all coils in that respective Group are activated, within all Blocks as well. In this manner, the permanent magnet position can always be defined as: (X_{i,j}, Y_{m,n}) where X signifies the X-direction Blocks and Groups, i ranges from 1 to 21 (# of repeated Blocks), and j represents the Group number from 1 to 3. Similarly. Y signifies the Y-direction Blocks and Groups, where m represents the Block number from 1 to 21, and n demonstrates the Group number from 1 to 3. At any given time, along any given pathway the permanent magnet can be defined in both the X and Y direction by a Block and Group. For example, each X-Block includes coils that each belongs to X-Group-1, X-Group-2 and X-Group-3 respectively. Each Y-Block includes coils that each belongs to Y-Group-1, Y-Group-2 and Y-Group-3 respectively. In FIG. 8B, it was demonstrated how the permanent magnet moves by stepwise motion and magnet motion is achieved by turning on the next EM coil in the desired direction of motion. This can be done simultaneously in any of the four cardinal directions. The magnet starts aligned with coils X-Group-2 and Y-Group-2. To move the magnet one step down, the previous Y-Direction coil would be switched off and the next coil powers on. Ergo, in Step-1 X-Group-2 remains on, while the Y-Group changes and Group-3 is now powered on. Likewise for Steps 2 & 3, the X-Group coils can be modulated in an identical manner to move the magnet along the X-Direction. In Step-3, it is noted that the magnet can be moved upward to realign with Y-Group-2. For both X and Y Directions the magnet can be moved in either a positive or negative direction along those axes. FIG. 8C shows a photo image of the EM PCB highlighting where the actual EM coils are located. More than 50% of the PCB area is dedicated to the EM coil array. This entire EM coil array is 70 cm×40 cm and located on the NAT PCB shown in FIG. 8C. When installed in the case, the magnetic robot rests on top of the PCB and the microfluidic cartridge racks underneath.

Second, the design is simulated within COMSOL to confirm the design style, understand the magnetic field, and to gauge appropriate distances. FIG. 8D shows a 2D planar view of the magnetic field generated by current inside one EM coil. 3D coil simulation demonstrated through four 2D slices along the length of the coil. The field distribution pictured matches our hypotheses that the strongest field gradient would pass through the center of the coil. Marked in red and blue are lines representing the distance from the coil that the permanent magnet would rest. The strongest sections of the magnetic field still encompass these distances and thus confirm the geometrical layout of our coil array.

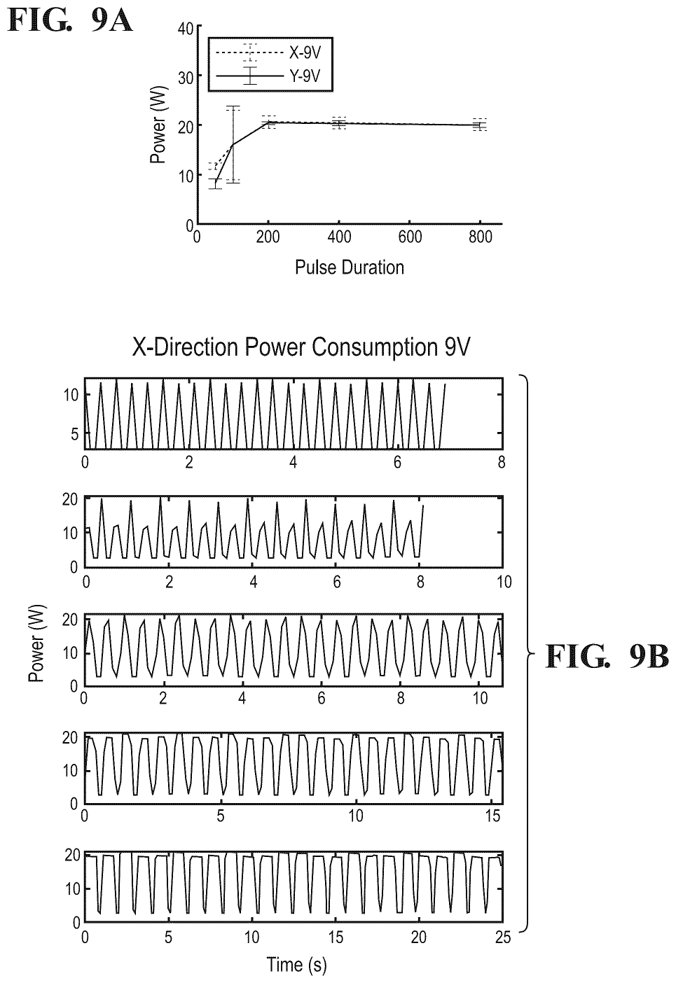

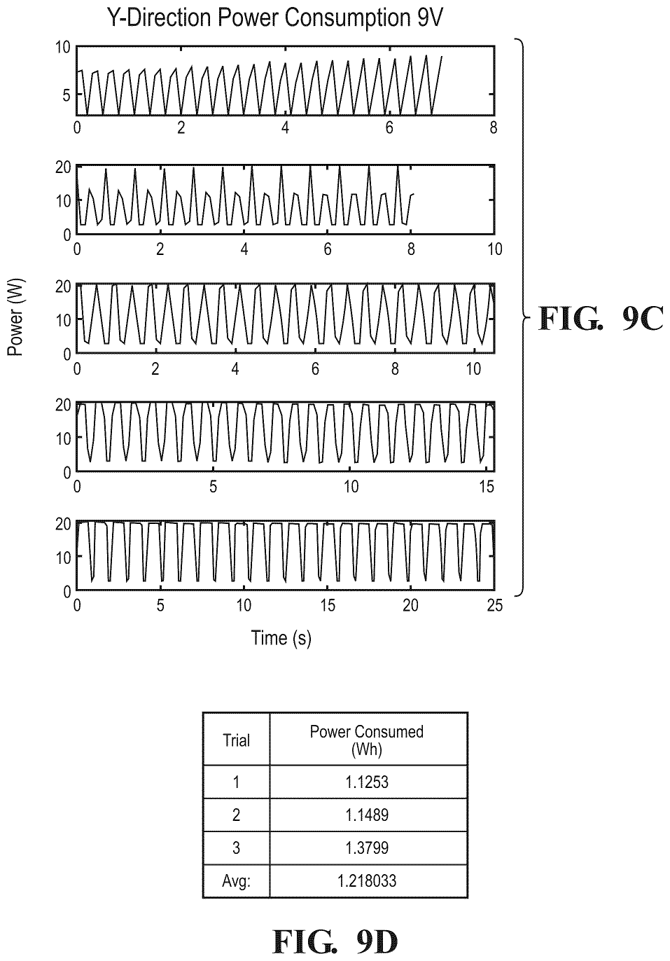

To test the accuracy of movement and minimize the power consumption of the NAT device, we examined the relationship between pulse duration and accuracy of the magnetic robot. Pulse duration is defined as the length of time for activation of one Group of EM coils. The relationship found can be seen in FIG. 8E. FIG. 8E shows accuracy of the magnetic robot's motion vs the length of time of activation for the EM coils in both X and Y directions. Motion in the Y-Direction and X-Direction demonstrate identical thresholds for when movement becomes highly accurate. Prior to this threshold of 200 ms we observed improper movement, small oscillations, and inactivity of the magnet. We hypothesize from our simulations and FIG. 8D that the magnetic field differences, therefore magnetic attraction forces, affect the inertia response of the magnetic robot. Next, we needed to examine the power consumption of the device under different pulse durations to select an appropriate size. FIGS. 9A-9D show device power consumption.

Peak power was recorded over a series of steps over three separate trials and is summarized in FIG. 9D, where all activation was kept at 1 Hz and averaged from 20 pulse commands seen in FIG. 9A. FIG. 9A shows Power per pulse for varying pulse durations. Frequency of activation was kept at 1 Hz and 25 pulses were recorded. Peak power begins to increase from 50 ms to 200 ms where it then flatlines at approximately 20 W per pulse. Raw data plots of power for each recorded trial starting from 50 ms (top) to 800 ms (bottom) are shown in FIG. 9B for X-Direction movement and in FIG. 9C for Y-Direction movement. Therefore, we can limit our power consumption and maximize accuracy by using a pulse duration of 200 ms for both X and Y directions. Therefore, for a 20 W pulse lasting 200 ms we expect one motion step to use 1.11 mWh (3.96 J). After determination of these parameters, the NAT device power consumption was tested during actuation of the magnetic robot for complete extraction movement. Summarized in FIG. 9D, the total device power consumption was recorded for three extraction procedures. The average power consumed during extraction was 1.21 Wh and occurred under 20 min. Thus, the entire NAT device consumes a reasonable amount of energy and can be powered by a 1600 mAh (14.4 Wh) LiPo battery. The NAT device introduces an automated extraction device that remains equipment-free and suitable for delivery in areas of low resource.

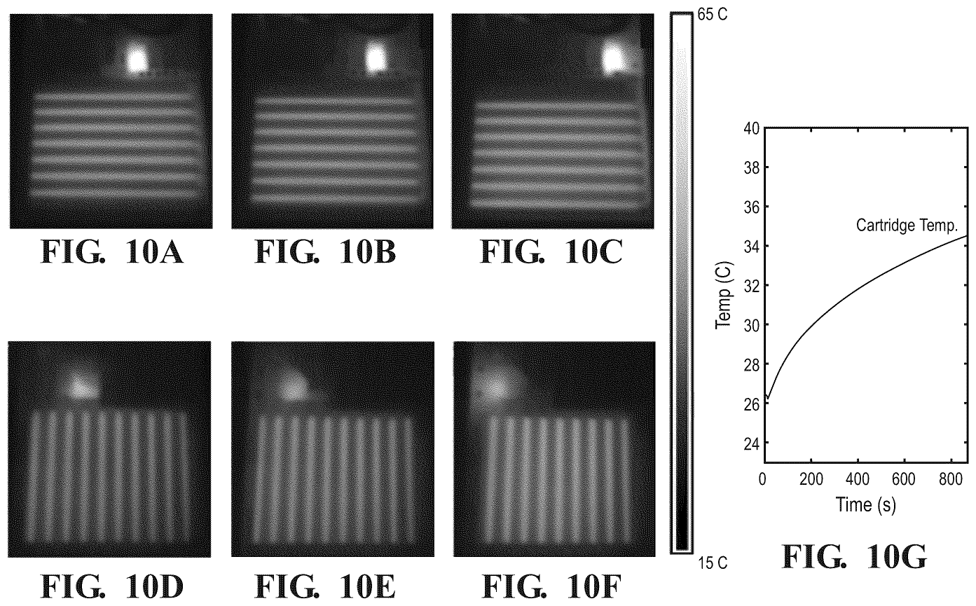

Another design benefit of the present EM coil setup is the ability for pulsed motion. This is important for reducing the amount of latent heat added to the enclosed device during EM coil operation. FIG. 8F shows temperature of the microfluidic cartridge over time when constant EM pulses are held at various frequencies. In FIG. 8F, we measured the temperature inside a cartridge when the NAT device was operated at difference constant frequencies of motion. The cartridge temperature can climb above 70° C. when the device operates at 2 Hz motion and above. However, when operated under 0.5 Hz the cartridge temperature does not climb above 40° C. The increased temperatures are conducted from the EM PCB that can become quite hot when actuated repeatedly or continuously. As an example, FIG. 10A-10F show thermal distribution and response of each X and Y coil after activation for 5 s. The thermal images show the distribution and geometry of the EM coils. Note that the MOSFET's are the hotspot in each image but after 5 s the coils and PCB do not reach excessive temperatures above 40° C. in under 5 s.

Next, we examined the temperature of the PCB during a mock extraction protocol. FIG. 8G shows thermal images of the PCB over time during an extraction protocol. Seen in FIG. 8G, the temperature of the PCB does increase over the 20 min runtime, but the temperature does not increase beyond 31° C. With the device closed, the cartridge temperature was tested during the duration of an actual extraction protocol where the max temperature never reached above 34° C. shown in FIG. 10G. The increase in temperature for the cartridges is likely due to the enclosed device retaining heat better than the bare PCB. Even so, we can be certain that the temperature inside the cartridge does not exceed human body temperature where our target RNA would. The present NAT device presents novel methods for magnetic bead manipulation that introduce programmable motion, redundant and accurate placement, reduced energy consumption, and reasonable heat dissipation.

### Computer Vision Magnetic Control Example

The following describes an exemplary computer vision magnetic control for an embodiment of the invention, but is only an example and is not limiting on other embodiments.

The magnetic robot is controlled and monitored using a computer vision algorithm to maintain its programmed pathway. Using charge switchable beads, the magnetic robot is capable of RNA extraction down to 1000 copies per sample. On the other hand, the magnetic robot with silica bead extraction exhibited RNA detection down to 100 copies per sample.

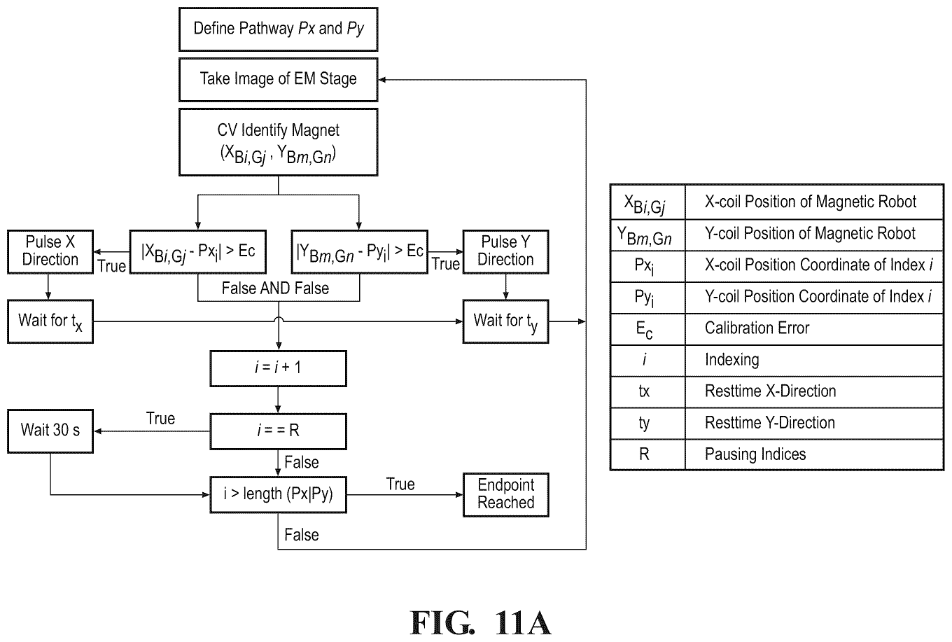

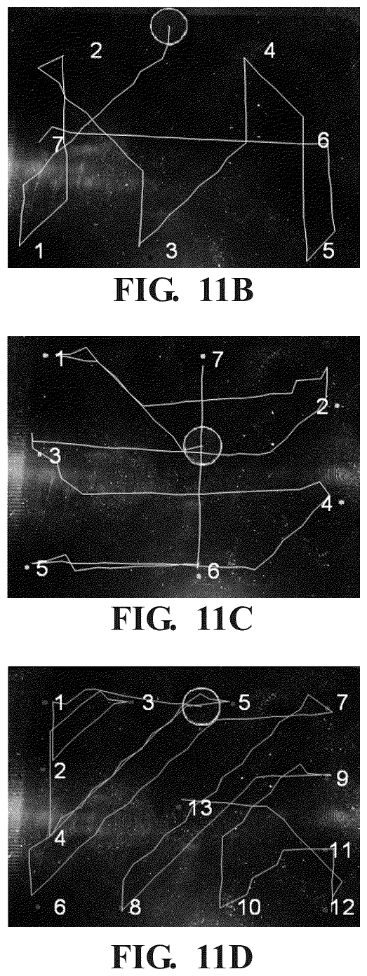

In the present device, Block and Group design of electromagnetic coils are introduced to create 2D motion from six activation circuits. However, for any given Group there are seven actuated coils across the 2D array. Therefore, to confirm the magnet location and control motion, a camera and computer vision algorithm is implemented to control the magnetic robot. The algorithm analyzes the magnetic robot position after each step and compares that position against preprogrammed locations. In FIG. 11A, the block diagram explains the logic flow through the camera vision algorithm for magnetic robot movement. Initially, a pathway for the magnetic robot is defined. This is only required once for each new cartridge configuration. Once imported, the program runs automatically for all future cases using the same cartridge. The input to the algorithm is an image of the magnet stage inside the device. From there the algorithm detects the location of the magnetic robot, compares the location, and will activate the appropriate EM coils to move the magnet in the direction that is needed to reach the set point.

First, an image of the magnet stage is captured with the internal camera. The magnetic robot is identified, and its position is defined as (X_{i,j}, Y_{m,n}). Then, the X and Y positions of the robot are compared against the first defined pathway point (Pₓ_{ᵢ}, P_{y}_{ⱼ}). If the magnetic robot needs to be moved in relation to this pathway point, then the appropriate EM coils by Group-# are actuated. The algorithm continues its feedback loop comparing the magnetic robot position (X_{i,j}, Y_{m,n}) to the pathway (Pₓ_{ᵢ}, P_{y}_{ᵢ}) where i=1. When the magnetic robot rests within the error margin of pathway point i (30 pixels), the algorithm indexes to the next pathway point, i=2. This process will repeat until the pathway point indices exceed the number of pathway points. At that time, the magnetic robot will be located at the last pathway point and the endpoint of the movement algorithm will have been reached. At that time, all the indication lights on the top of the NAT device will pulse, indicating to the user that the cartridge is ready for removal.

To demonstrate that the algorithm is integrated with the EM coils, three unique pathways were defined for the magnetic robot to follow. FIGS. 11B-11D show photo images of the EM PCB overlayed with the movement pathway of the magnetic robot for the numbered pathway. The magnetic robot can be moved in any of the four cardinal directions, can accurately follow a pathway, and is capable of tracking various pathways. These pathways, seen in FIGS. 11B-11D, exaggerate movement in the four cardinal directions as proof of motion. In FIG. 11B, a pathway mimicking a sine wave propagates along the +X direction. FIG. 11C demonstrates a sine wave propagating along the −Y direction, and FIG. 11D defines a combination of the two prior pathways. These images show the tracked pathway that the magnetic robot followed to reach its target points. The controlled and programmable motion in 2D space presents an approach to magnetofluid manipulation. These methods present magnetic bead manipulation for any number of applications even beyond nucleic acid extraction. The NAT device presents a handling system capable of various cartridge control patterns and is capable of handling complicated cartridge designs. Herein, we will demonstrate the capabilities of the NAT device to extract HIV viral RNA from buffer and plasma as proof of concept.

### HIV RNA Extraction Example

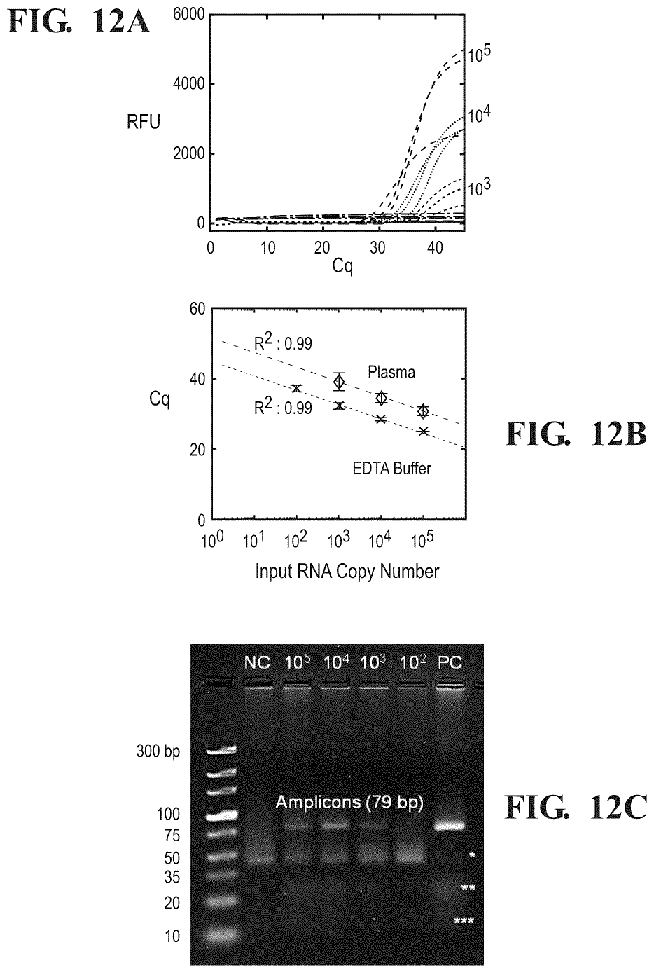

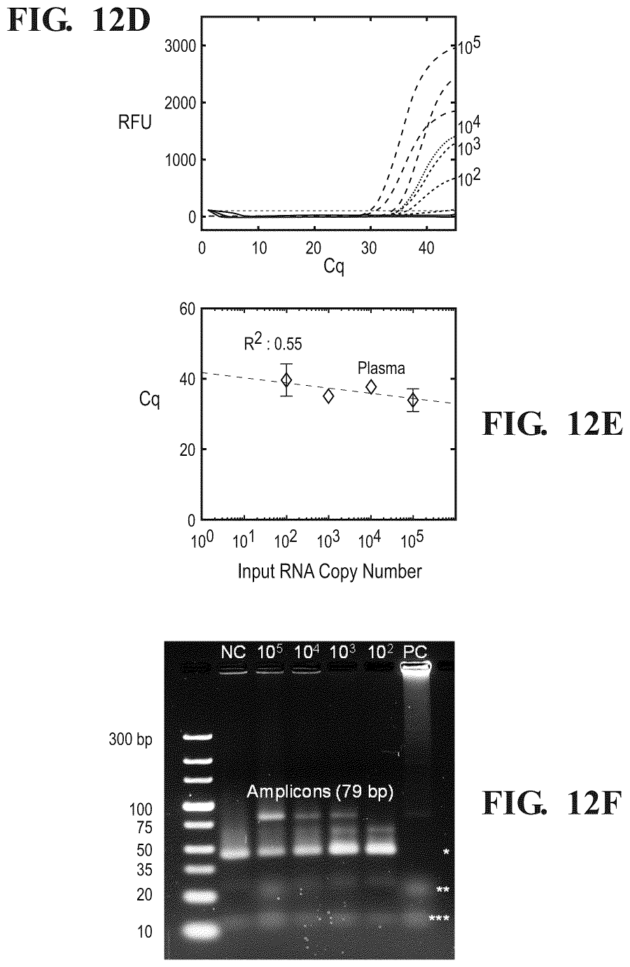

To demonstrate the performance of the NAT device and microfluidic cartridge, we tested the protocol with spiked samples of oligo RNA into EDTA buffer (Ethylenediaminetetraacetic acid) and healthy human plasma. ChargeSwitch RNA extraction from contrived buffer and plasma samples was entirely operated by the ProMagBot device. Following extraction, all samples were immediately frozen at −80° C. for later testing by RT-qPCR. FIGS. 12a-12F show plasma extraction performance using the present NAT device and protocol. As seen in FIG. 12A, the extraction protocol could extract synthetic RNA copies from 10⁵ down to 10³ for plasma samples. Thus, demonstrating the ability of the device to handle low nucleic acid copies inside a small sample volume. In FIG. 12B, the amplification performance for EDTA buffer and plasma shows a ˜10% difference in C_{q} value. Extraction from EDTA buffer could be achieved down to 10² input copies. The added complexity of the plasma matrix compared to buffer likely caused the degradation in performance through poor bead binding or increased carryover debris. The increased amounts of proteins, solutes, and other debris in plasma could be carried over to the elution buffer where it can inhibit and degrade the target RNA. Despite the disparity in C_{q} value, extraction from both sample media demonstrated strong linearity, shown in FIG. 12B. Therefore, the NAT device could be used for upstream preparation of quantitative analysis.

Next, gel electrophoresis verified the amplicons present, checked for any undesired carryover nucleic acids, and confirmed our extraction from PCR amplification. As seen in FIG. 12C the gel image shows DNA bands with identical results to our PCR analysis where detection is absent in samples containing 10² input copies.

First, note the region of high contrast at the amplicon band length of 79 bp. Second, note the faint band at the bottom signifying the short primers. Third, note the presence and absence of the amplicon band from the positive-control (PC) and negative-control (NC) wells respectively. The amplicon band can be seen at 79 bp and below that the primers (forward and reverse) and probes can be vaguely distinguished, respectively. From these results we can be confident that the detection assay works as expected and that the extraction procedure is compatible with downstream PCR analysis.

Last, to increase the overall application of the NAT device and offer an improvement on the limit of extraction, we integrated another PMB extraction assay into our design, MagMAX beads. For this to work, the microfluidic cartridge had to be redeveloped to handle the MagMAX beads, lysate volume, and simplified reagents. For extraction with MagMAX beads, we were forced to simplify their recommended reagents to eliminate ethanol and isopropanol. These reagents react strongly with our PMMA cartridge and sealing methods. However, once developed, the cartridge and the NAT device were able to demonstrate extraction of HIV RNA from plasma samples, shown in FIGS. 12D and 12E. The reduction of reagents was a compromise in extraction performance where linearity and consistency were lacking, shown in FIG. 12E, the NAT device was able to extract RNA copies 10⁵ down to 10²; however, several samples showed no extraction and the linear performance of those that were, was poor. We saw similar results in the gel analysis to the ChargeSwitch kit except with more smearing, shown in FIG. 12F. This likely is caused by introduction of carrier RNA, adding more non-specific sequences, and/or genomic DNA carry-over. We suspect that the removal of ethanol and isopropanol from the assay caused decreased performance due to poor nucleic acid precipitation from the lysate. Also, our microfluidic cartridge required reduced reagent volumes that likely reduced performance because of improper chemical ratios. Despite these simplifications, we still demonstrated compatibility with other magnetic bead extraction assays and the ability to modify, adapt, and program the NAT device for another extraction assay. We hypothesize that our performance limit (100 copies) and repeatability could be improved through optimization of these extraction assays. The compatibility of our PMMA cartridge with the required reagents can be solved through other methods of fabrication (3D printing or injection molding) with inert materials.

### Copy Number Sensitivity of HIV-1 RT-LAMP Test

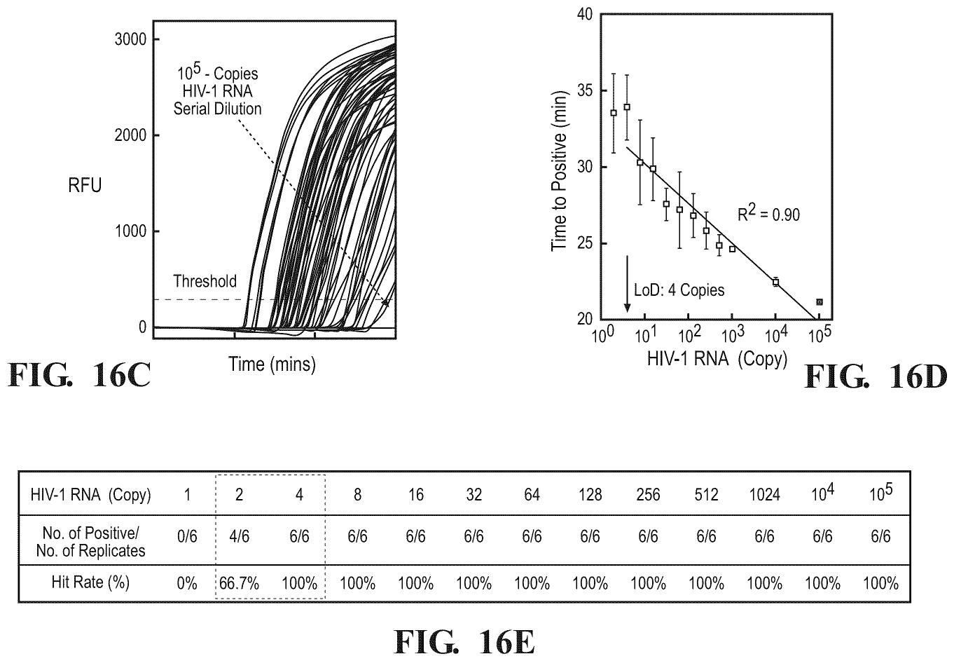

We used a previously validated HIV-1 LAMP primer set against the highly conserved region of the integrase gene within subtype B (Curtis et al. 2012) with a modified fluorescent reporter of Calcein (Tomita et al. 2008). We first validated the intrinsic copy number sensitivity of the HIV-1 RT-LAMP assay by performing the RT-LAMP reaction against the quantitative panel of HIV-1 RNAs at concentrations ranging from 10⁵ copies/μL down to 1 copy/μL. FIGS. 16A-16F show validation of the HIV-1 RT-LAMP assay with the quantitative panel. FIG. 16A summarizes the RT-LAMP primers. FIG. 16B shows the RT-LAMP reaction setup. FIG. 16C shows the real-time RT-LAMP amplification data with serially diluted HIV RNA standards. Each concentration was repeated six times. As shown in FIGS. 16C-16E, the copy number sensitivity of the HIV-1 RT-LAMP was determined to be four copies. Changes in temperature or storage time can affect the performance of LMAP. The prepared LAMP should be kept in a refrigerated environment, and it is best to use it immediately. FIG. 16D shows time to positive value at different HIV-1 RNA concentrations. The time to positive is defined as the time needed for the RFU to reach the threshold level of 300 (dashed line in c). FIG. 16E shows summary of the hit rate. All six reactions with four or more copies of RNAs can be amplified successfully. The copy number sensitivity of the HIV-1 RT-LAMP was determined to be four copies.

### Whole Blood HIV-1 RT-LAMP Assay Test

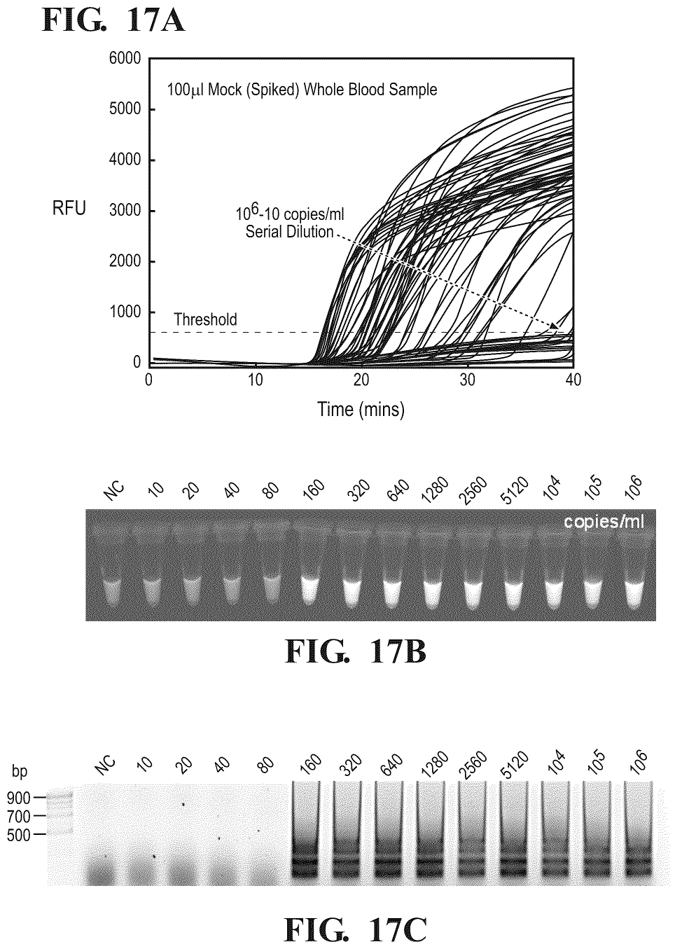

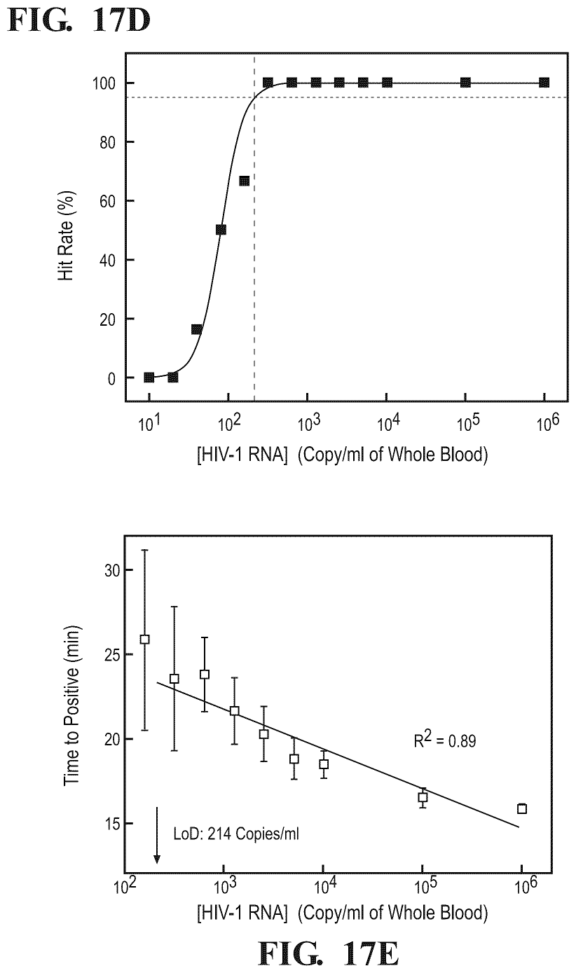

To further test the impact of the whole blood matrix and the reagent on the HIV-1 RT-LAMP assay, we formed mock HIV-1 positive samples by spiking the HIV-1 RNA into healthy whole blood. The 100 μL of mock samples at concentrations from 10 to 10⁶ copies/mL, were mixed with 500 μL lysis buffer, 200 μL, binding buffer, and 15 μL charge switchable magnetic beads for lysis and binding. The beads were then washed with 450 μL of washing buffer. The RNAs were directly eluted into a 25 μL master mix for RT-LAMP reaction. FIGS. 17A-17E show RT-LAMP assay validation with spiked whole blood mock sample. FIG. 17A presents the real-time RT-LAMP results (each concentration was repeated six times). FIG. 17B shows the fluorescent image of the reaction tubes under the ultraviolet (UV) light. FIG. 17C shows the gel electrophoresis results in 2% agarose gel, in which clear ladder-like patterns with multiple bands of different molecular sizes were observed due to the stem-loop DNA structures with several inverted repeats within LAMP amplicons (Notomi et al. 2000; Tomita et al. 2008). The fluorescent image and the gel images agreed well with each other.

To estimate the LoD of whole blood HIV-1 RT-LAMP assay, we examined the hit rates at different RNA concentrations (Holstein et al. 2015). The hit rate is defined as the number of amplified samples over all samples. As shown in FIG. 17D, the LoD of the whole blood RT-LAMP assay is determined to be 214 copies/mL at the 95% confidence level. This LoD is higher than that obtained with the HIV-1 quantitative panels (4 copies, FIG. 16E). We believe the following factors are responsible for the deteriorated LoD in the whole blood samples. First, although we used the spiked sample as soon as we prepared it, the HIV-1 RNA can still experience a certain degree of degradation in the whole blood. Second, inhibitors could exist with the whole blood sample. Third, there is a possibility of material loss during the sample preparation process. We further examined the time to positive as a function of the RNA concentrations in whole blood. As shown in FIG. 17E, linear fit produced the R² with 0.89, similar to those obtained with the HIV-1 quantitative panels, shown in FIG. 16D.

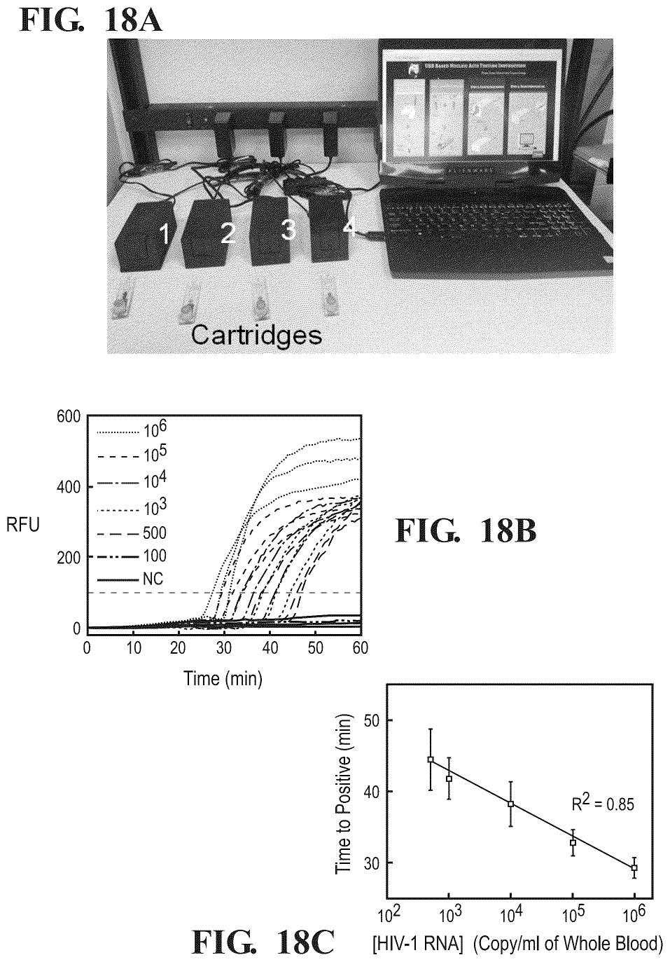

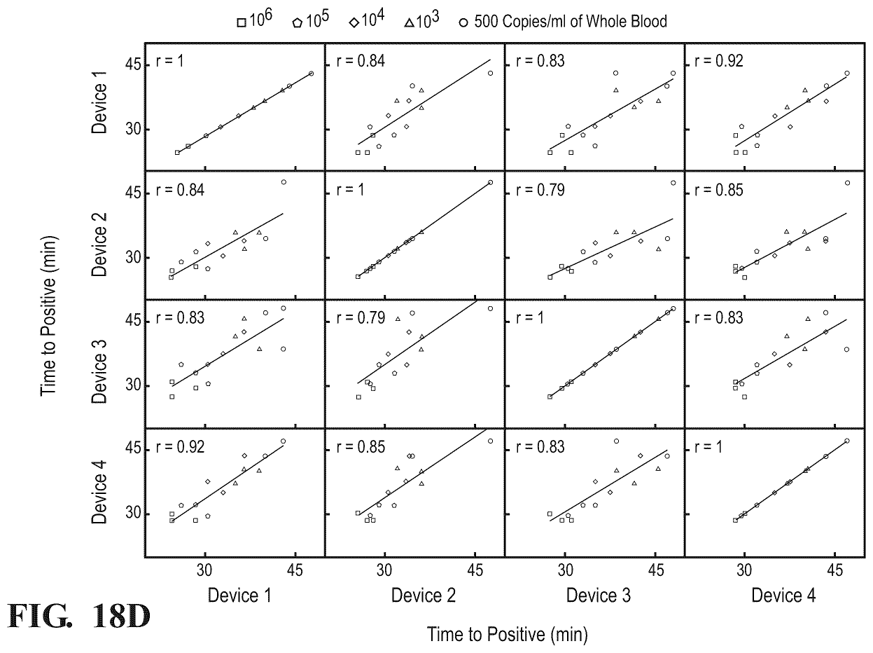

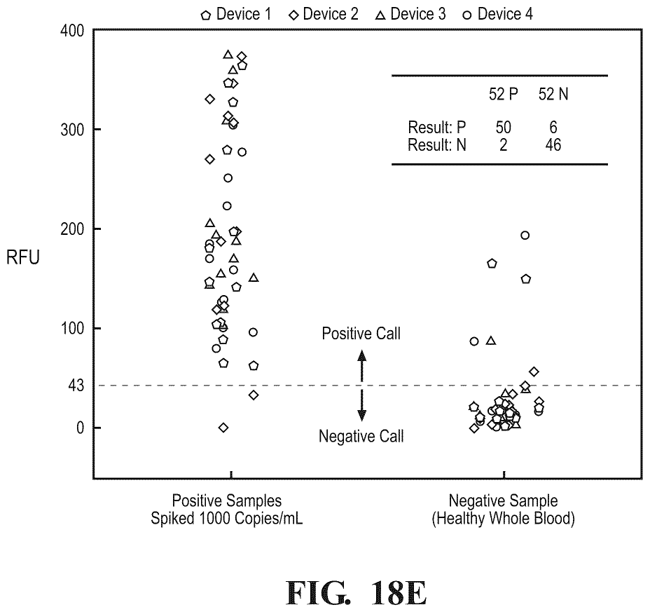

After validating the automated sample preparation and the HIV-1 RT-LAMP assay, we went out to test the intra- and inter-device performances. It is noteworthy that the multiple USB-interfaced analyzers can be used simultaneously and independently in a plug-and-play (PnP) fashion, shown in FIG. 18A.

For the intra-device verification, we tested a series of mock samples with different HIV-1 RNA concentrations. FIG. 18B shows the real-time data obtained from testing a triplicate panel of these samples with a single USB interfaced analyzer. As shown, HIV-1 RNA concentrations at 500 copies per mL of whole blood were all amplified successfully, in par with these obtained in the tube, shown in FIG. 17D. FIG. 18C shows the time to positive as a function of the input RNA concentrations. A linear fit produced the R² with 0.85, indicating the feasibility of using the USB-interfaced analyzer for a semi-quantitative test on the whole blood (i.e., differentiating between high, medium, and low viral load).

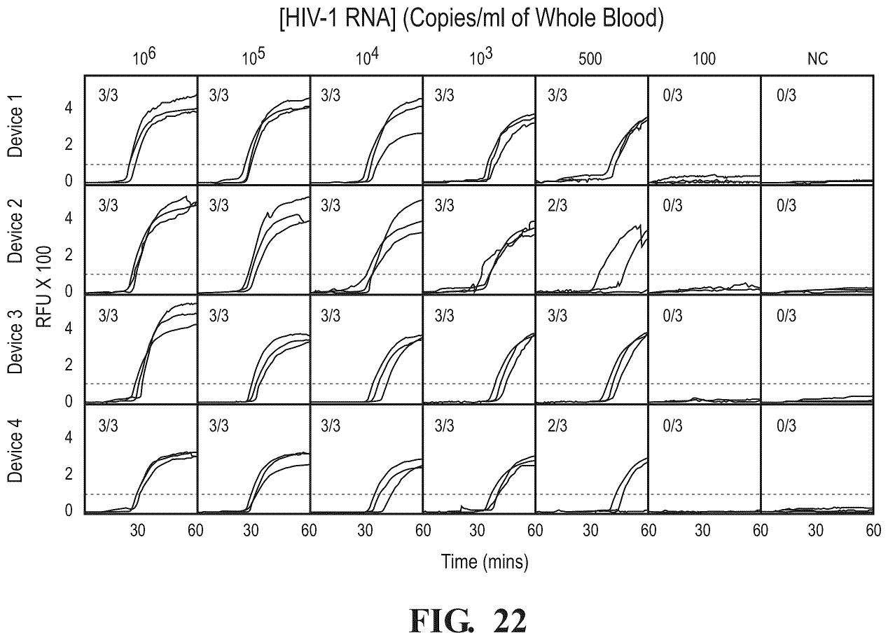

For the inter-device verification, we tested four independent devices with multiple triplicated mock samples. FIG. 22 summarizes the real-time data obtained from these tests. FIG. 22 shows the real-time RT-LAMP curves for the inter-device test (4 devices) at different HIV-1 RNA concentrations in whole blood. The hit rate for RNA concentrations above 1000 copies/mL is 100% for all devices tested. The hit rate dropped to 83% (10/12) with samples of 500 copies/mL. No amplification curves were observed for samples at 100 copies/ml, and negative control samples;

We benchmarked the time to positive between any two devices and examined their Pearson correlation coefficient. As shown in FIG. 18D, the device to device showed Pearson correlation coefficients ranging from 0.79 to 0.92, suggesting a good quantitative agreement between these devices.

To determine the diagnostic ability of the HIV NAT-on-USB, we tested a total of 104 whole blood samples (52 negatives and 52 positives) with four different analyzers. The 52 positive samples were constructed by spiking the HIV-1 RNA into 100 μL human whole blood to form a concentration of 1000 copies/mL, a clinically relevant viral load threshold used for routine monitoring of HIV in resource-limited settings (Ellman et al. 2017; Manoto et al. 2018). We examined the fluorescence values for all samples at 60 min, shown in FIG. 18E. As shown, the RFU values of positives were significantly higher than that of the healthy controls. To find the optimal fluorescence threshold to differentiate the positives and negatives, we analyzed the receiver operating characteristic (ROC) curve (Bewick et al. 2004; Zweig and Campbell 1993) by varying the threshold from 1 to 500 RFU. In general, increasing the threshold will improve the specificity but deteriorate the sensitivity. The optimal RFU threshold from ROC analysis is 43 (dashed line in FIG. 18E). The inset of FIG. 18E summarized the diagnostic performance with this optimized threshold. 50 out of 52 positives were detected as true positives, and 46 out of 52 negatives were detected as true negatives. The sensitivity, and specificity of the test was 96.2% (95% CI=90.9%-100%) and 88.5% (95% CI=79.8%-97.1%). The tests performed with all four different devices showed excellent accuracy (93%) in differentiating the clinically relevant viral load threshold at 1000 copies/mL.

### Exemplary Materials and Methods

The following describes exemplary materials and methods for an embodiment of the invention, but is only an example and is not limiting on other embodiments.

### 1. Microfluidic Cartridge

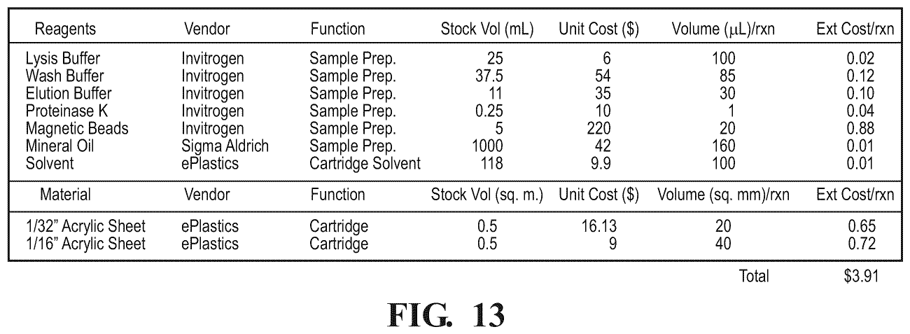

Inside the microfluidic cartridge there are 4 separate components. The three main chambers house the lysate mixed and loaded from the user. 80 (uL) of washing buffer), and 30 (uL) an elution buffer. The last remaining component is mineral oil (80 uL x2) that separates the chambers (MilliporeSigma, USA). The cartridge itself is composed of three stacked layers: base, channel spacer, and cover. These layers are 1/32″, 1/16″, and 1/32″ thick respectively, of polymethyl methacrylate (PMMA) purchased from Inventables.com. These layers are designed using Creo Parametric (PTC, USA) and then laser-cut using a VLS3.60DT from Universal Laser Systems (Scottsdale, AZ). The three separate layers are bonded together using an acrylic solvent and treated under UV-light for 60 minutes for disinfection. All reagents and materials used are detailed in FIG. 13.

### 2. Cartridge Adjustments for MagMAX Kit

The MagMAX cartridge holds the same footprint as the original microfluidic cartridge with modifications made to the channel width and lysis chamber size. The channel width was increased from 2 mm to 3 mm and the lysis chamber was increased in size to hold 150 μL of lysate. All other design and fabrication methods are identical to the original cartridge.

### 3. NAT Device and EM PCB

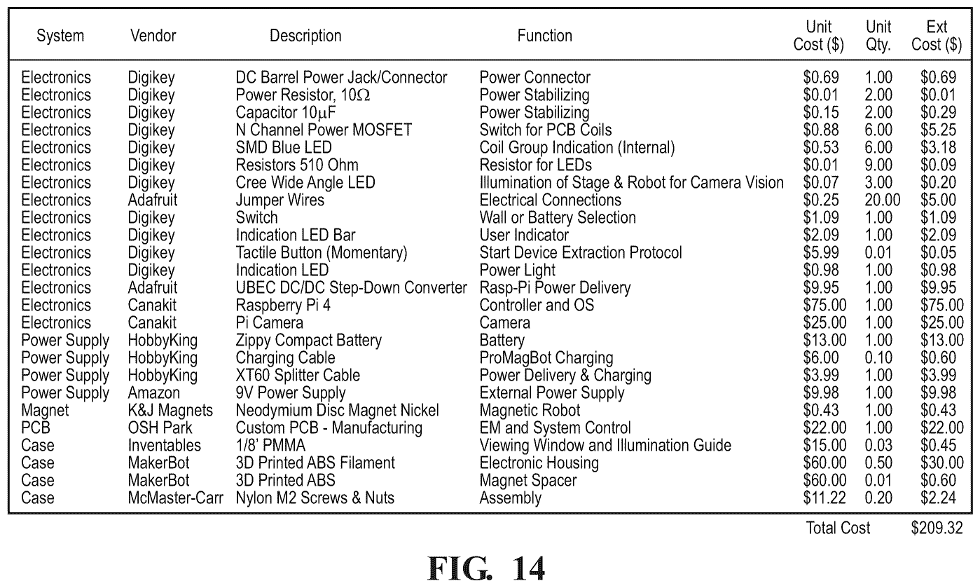

All components of the NAt device are housed within a 3D printed case made of ABS plastic. The 3D printer and ABS material are sourced from MakerBot (MakerBot Industries, LLC). The case, spacers, and viewing windows were modeled using Creo Parametric. The customized EM PCB and control board were designed in AutoCAD Eagle (AutoDesk Inc.) and fabricated by OSH Park LLC (Portland, OR). The magnetic robot is a permanent magnet from K&J Magnets (Pipersville, PA) with a 0.25″ diameter and magnet density of N55. All other various electronic components: indication LEDs, push buttons, switches, or connectors, were all purchased from Digi-Key, shown in FIG. 14. The LiPo battery, XT60 connector, and JST-XH connectors were purchased from HobbyKing (Hong Kong). The 9 V 6 A Power Supply used as an option for connecting the ProMagBot device to a wall outlet was purchased from Amazon (Seattle, WA). The Raspberry Pi 4 and Pi Camera module v2 were purchased from Cana Kit (North Vancouver, Canada).

### 4. Computer Vision

Computer vision and magnetic robot tracking is accomplished by using the Raspberry Pi camera module v2 paired with a Raspberry Pi 4. The module is mounted 3.5 in. away from the magnetic robot and stage. The stage is illuminated by three 120-degree, wide angle LEDs that illuminate the viewing window within the device. Image capture and MagBot detection is automated by a custom Python script on the Pi 4.

### 5. COMSOL Simulation

Magnetic field simulations were run within COMSOL 2020 (COMSOL Inc.). Our model was adapted from the stock Electromagnetic coil simulation and the geometry of our PCB coil was imported into the software. The current in the simulation was set as 1.5 A and the boundary around the coil was defined as a 2 cm×2 cm×7 cm rectangular prism.

### 6. Automated Extraction

To prepare the NAT device for automated extraction, an extraction pathway was calibrated on the device to match the computer vision placement of the permanent magnet with the actual response of the magnetic beads in solution below. This calibration is only required once for each cartridge geometry to establish pathway locations. Once complete, the device recalls those saved locations from a .csv file and automates the rest of the extraction without need for user input.

### 7. Plasma Extraction

Prior to being spiked with synthetic RNA (Integrated DNA Technologies, IA, USA), healthy plasma samples were pretreated with 1 μL of proteinase K (Invitrogen, USA) and incubated at 60° C. for 15 minutes. Each 50 μL sample was then spiked with RNA from 10⁵ to 10⁰ copies. The plasma sample was then combined with a premixed solution of ChargeSwitch beads (10 μL), Lysis Buffer (20 μL), and Binding Buffer (19 μL) all from the ChargeSwitch Total RNA Extraction Kit (Invitrogen, USA). Once combined the total volume of 100 μL was inserted into the lysis chamber of the microfluidic cartridge. All air inlets on the cartridge were pre-sealed with tape and the cartridge can be inserted into the ProMagBot device for automated extraction.

When using the MagMAX extraction kit (Invitrogen, USA), the lysis chamber volumes were 20 μL of MagMAX beads, 15 μL of binding enhancer, Lysis 40 μL, Isopropanol 40 μL, Carrier RNA 2 μL. The washing chamber still used the ChargeSwitch Wash, and the elution chamber held 30 μL of MagMAX elution buffer.

### 8. RT-qPCR Assay

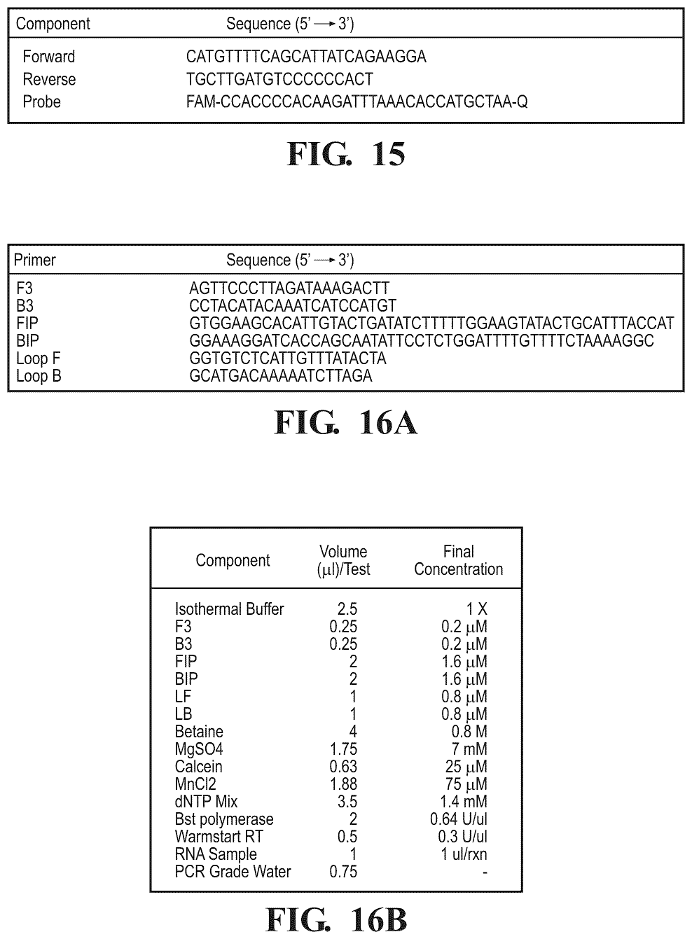

Procedure and reagents followed the PCR HIV assay validated by Palmer et. al. FIG. 15 [62]. From 30 μL of elution buffer, 10 μL was used as RNA sample for PCR analysis. Therefore, our total PCR volume consisted of 25 μL: 6.25 μL of Fast Taq One-Step Master Mix (Applied BioSciences, USA), 1.5 μL of Forward and Reverse primer, 0.63 μL of probe, 10 μL of extracted RNA sample, and 5.13 μL of Nuclease-free water (New England Biosciences, USA). Testing was performed on a BioRad C1000 Thermal Cycler (Hercules, CA). Thermal Cycling was set as: 50° C. for 5 min, 95C for 3s, 65C for 30s repeated 45× [62]. Primers and probes were purchased from Integrated DNA Technologies (Coralville, IA).

### 9. Gel Electrophoresis

Gel electrophoresis was completed using an agarose gel of 5% wt. Gel was made using agarose powder and 50X TBE buffer that was doped with SYBR Safe Stain (Invitrogen, USA) and cured for 45 min. The DNA ladder was Thermofisher's Ultra Low DNA Ladder (Thermofisher, USA). Each gel lane was loaded with 15 μL of PCR and loading buffer with a ratio of 5:1. The voltage was set to 110 V and ran for 50 min. before imaging on BioRad's GelDoc Go (Hercules, CA).

### 10. Data Analysis Method and Statistics

All statistical analysis and regression modeling was completed using MATLAB R2020. All plots showing data demonstrate mean and 3 SD for triplicate testing unless otherwise noted. All data processing was handled within MATLAB. All figures and plots were created with MATLAB and PowerPoint.

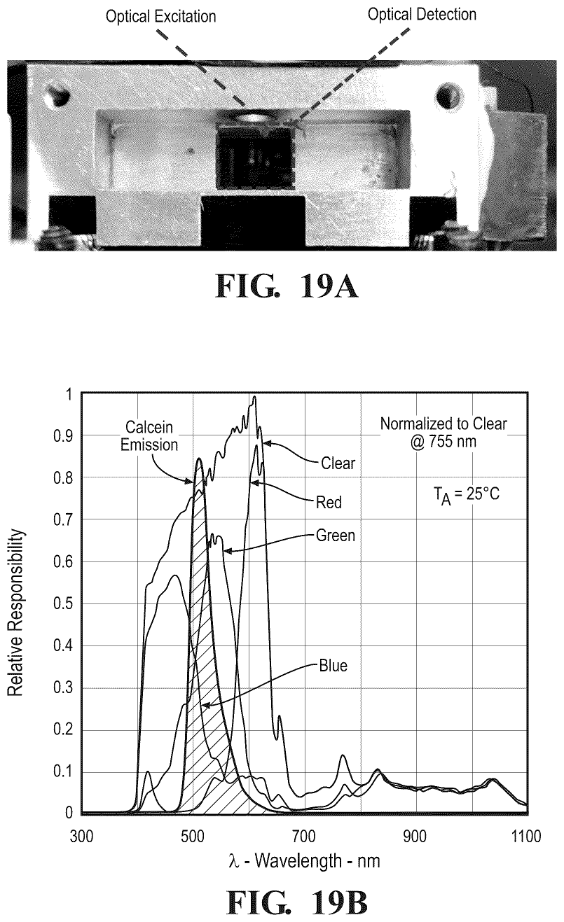

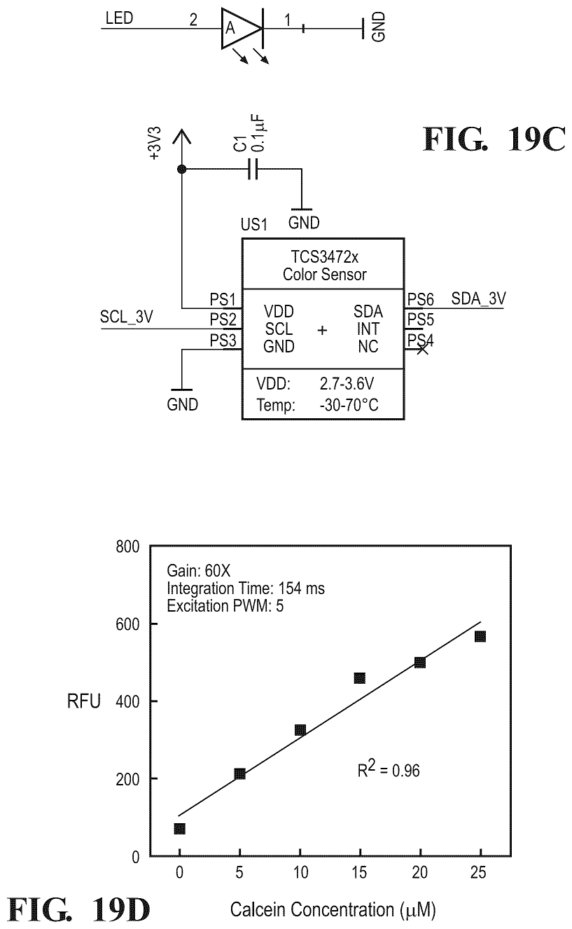

After assembly of the device in FIGS. 1A and 1B, we validated that the optical sensor has a linear response to the Calcein concentration from 0 to 25 μM, shown in FIGS. 19A-19D, confirming its suitability for real-time monitoring of the amplification process. FIG. 19A shows a photo image of the optical module. On the excitation side, an LED light source (λ=488 nm) illuminates the LAMP reaction chamber. On the detection side, the emitted light from the LAMP reaction chamber is guided to the optical sensor through PMMA. The incident direction of the excitation LED light is perpendicular to the optical sensor to minimize the diffracted excitation light entering the optical sensor, thereby improving the signal-to-noise ratio. FIG. 19B shows optical responsibility of the optical sensor as well as the calcein emission profile. The optical sensor can collect the signals from the red, green, and blue channels. The analyzer uses the red channel of the optical sensor to monitor the emission signal. FIG. 19C shows the circuit schematic diagram of the excitation and emission sensing module. FIG. 19D shows characterization of the optical sensor with calcein. The optical sensor showed a linear response to the concentration of Calcein from 0 to 25 μM. The test is performed with gain 60×, the integration time 154 ms, and PWM of the excitation LED control is 5.

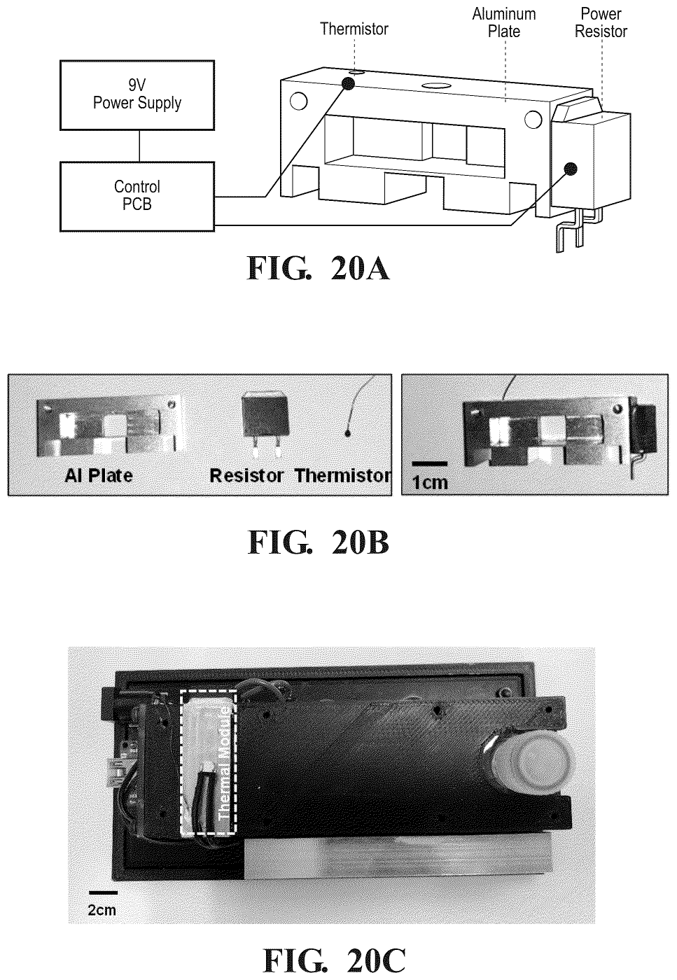

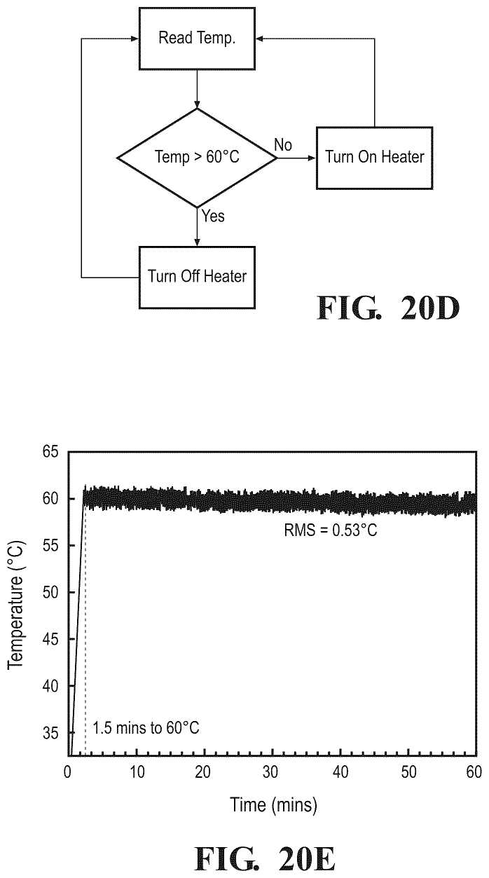

In addition, we validated that the resistive heating module can reach the desired 60° C. within 1.5 mins and the root mean squared (RMS) value of the temperature is 0.53° C. after stabilization, shown in FIGS. 20A-20E, which can meet the temperature requirement of the LAMP assay (Rudolph et al. 2015). FIG. 20A shows the thermal module is composed of a power resistor as a heating source, a thermistor as a temperature feedback sensor, and a CNC aluminum plate. FIG. 20B shows a photo image of each component in the heating module (left) and the assembled heating module (right). FIG. 20C shows a photo image of the thermal module assembled on the analyzer (top view). FIG. 20D shows a flow chart of the thermal control algorithm. FIG. 20E shows the thermal module can reach the required 60° C. within 1.5 minutes, and the root mean square (RMS) value of the temperature after stabilization is 0.53° C., which can meet the temperature requirements of LAMP detection;

As will be clear to those of skill in the art, the embodiments of the present invention illustrated and discussed herein may be altered in various ways without departing from the scope or teaching of the present invention. Also, elements and aspects of one embodiment may be combined with elements and aspects of another embodiment. It is the following claims, including all equivalents, which define the scope of the invention.

1. A nucleic acid testing device, comprising:
a microfluidic cartridge having a binding chamber, a washing chamber and a reaction chamber;
an analyzer having a mechanical module, the mechanical module including an electromagnetic coil array and a permanent magnet, the electromagnetic coil array generating a programmable pulsed electromagnetic field to actuate the permanent magnet that further controls a paramagnetic bead (PMB) assay in the cartridge; and
a processing unit for controlling the analyzer.

2. The nucleic acid testing device according to claim 1, wherein the electromagnetic coil array is a double-sized planar electromagnetic coil array.

3. The nucleic acid testing device according to claim 1, wherein the electromagnetic coil array is a linear array.

4. The nucleic acid testing device according to claim 1, wherein the electromagnetic coil array is a 2D array organized in blocks and groups.

5. The nucleic acid testing device according to claim 4, wherein each block holds three groups, each group having a group designation, coils with a same group designation are interconnected across blocks.

6. The nucleic acid testing device according to claim 1, wherein the electromagnetic coil array and the permanent magnet are located above or under the microfluidic cartridge.

7. The nucleic acid testing device according to claim 1, wherein the microfluidic cartridge further comprises valve chambers located between the binding chamber, the washing chamber and the reaction chamber.

8. The nucleic acid testing device according to claim 1, wherein the microfluidic cartridge further comprises air traps and inlet ports.

9. The nucleic acid testing device according to claim 1, wherein the analyzer further comprises an optical module for excitation/detection and a thermal module for actuation/sensing.

10. The nucleic acid testing device according to claim 1, further comprising a battery for powering the nucleic acid testing device.

11. The nucleic acid testing device according to claim 1, further comprising a housing in which the cartridge and analyzer are disposed.

12. A method of nucleic acid testing, comprising the steps of:
providing a nucleic acid testing device, comprising:
a microfluidic cartridge having a binding chamber, a washing chamber and a reaction chamber;
an analyzer having an electromagnetic coil array paired with a permanent magnet;
providing a paramagnetic bead (PMB) assay bound to nucleic acids forming nucleic acid-bead complexes;
driving the nucleic acid-bead complexes into the washing chamber using the permanent magnet;
removing unwanted proteins and salts carried along with the nucleic acids in the washing chamber;
moving the nucleic acid-bead complexes into the reaction chamber using the permanent magnet for elution where the nucleic acids dissociate from the PMBs; and
moving the PMBs away from the reaction chamber using the permanent magnet, leaving the nucleic acids in the reaction chamber for downstream analysis.

13. The method according to claim 12, further comprising programming the electromagnetic coil array to generate a localized pulsed electromagnetic field to control the permanent magnet.

14. The method according to claim 12, wherein the electromagnetic coil array is a linear double-sized planar electromagnetic coil array.

15. The method according to claim 12, wherein the electromagnetic coil array is a 2D array organized in blocks and groups and activated in series.

16. The method of claim 15, wherein each block holds three groups, each group having a group designation, coils with a same group designation are interconnected across blocks, coils are activated by group designation.

17. The method according to claim 12, wherein beads in the assay are horizontally agitated by a programmed electromagnetic sequence.

18. The method according to claim 12, wherein the nucleic acid is HIV, HPV, HBV, HCV RNAs.

19. The method according to claim 12, wherein the nucleic acid is a DNA.

20. The method according to claim 12, wherein the sample is blood, plasma, urine, saliva, or virus transfer medium.
